(19) United States
(12) Patent Application Publication
(10) Pub. No.: US 2012/0157387 A1
(43) Pub. Date: Jun. 21, 2012

(54) ORALLY BIOAVAILABLE D-GAMMA-GLUTAMYL-D-TRYPTOPHAN

(75) Inventors: Tim Fat TAM, Vaughan (CA); Regis Leung-Toung, Mississauga (CA); Yingsheng Wang, Toronto (CA); Yanqing Zhao, Richmond Hill (CA); Tao Xin, Vaughan (CA); Wanren Li, Toronto (CA); Jolanta Maria Wodzinska, Mississagua (CA); Vrajlal S. Rabadia, Mississauga (CA); Birenkumar Shah, Brampton (CA); Christopher John Feeney, Toronto (CA)

(73) Assignee: APOTEX TECHNOLOGIES INC., Toronto (CA)

(21) Appl. No.: 13/400,351

(22) Filed: Feb. 20, 2012

Related U.S. Application Data
(63) Continuation-in-part of application No. 12/515,217, filed on May 15, 2009, now Pat. No. 8,119,606.

(30) Foreign Application Priority Data
Nov. 28, 2006 (CA) 2569204

(51) Int. Cl. A61K 38/05; A61P 17/06; C07K 5/072

(52) U.S. Cl. 514/18.6; 548/491

(57) ABSTRACT

Provided are compounds which are prodrugs of D-gamma-glutamyl-D-tryptophan, methods of making the compounds and methods for using the compounds.

### TECHNICAL FIELD

This invention relates to the field of prodrugs of dipeptides and more particularly to the field of prodrugs of the dipeptide D-gamma-glutamyl-D-tryptophan (H-D-Glu(D-Trp-OH)—OH).

### BACKGROUND

A prodrug is a compound that is modified in the body after its administration to provide an active drug. Depending on the therapeutic use and mode of administration, a prodrug may be used orally, for injection, intranasally, or in an inhaler formulation directed at lung tissues (Rautio et al. Nature Reviews Drug Discovery 7, 255-270 (February 2008). The use of prodrug compounds in an inhaler formulation directed at the lung tissue has been reviewed (Proceedings Of The American Thoracic Society Vol 1 2004, How the Lung Handles Drugs, Pharmacokinetics and Pharmacodynamics of Inhaled Corticosteroids, Julia Winkler, Guenther Hochhaus, and Hartmut Derendorf 356-363; H. Derendorf et al., Eur Respir J 2006; 28: 1042-1050).

For inhaler and intranasal means of administration, the minimization of oral bioavailability and systemic side effects by rapid clearance of absorbed active drug may be some of the design considerations. A prodrug designed for oral administration may prefer an improvement to oral bioavailability upon oral administration to animals, and appropriate chemical stability in simulated digestive fluids at pH 1.2 (also known as simulated gastric fluids) or pH 5.8 or 6.8 (also known as the simulated intestinal fluids). For prodrugs that are used an in injection, the aqueous solubility of the compound is an important consideration.

The screening criteria for prodrugs depend on its mode of administration. However, a prodrug that can be readily hydrolyzed to the active drug in a human blood is a positive feature upon administration. Human blood has esterases that are capable of biotransforming some ester derivatives to the active drug (Derek Richter and Phyllis Godby Croft, Blood Esterases, Biochem J. 1942 December; 36(10-12): 746-757; Williams F M. Clinical significance of esterases in man. Clin Pharmacokinet. 1985 September-October; 10(5):392-403). In addition, prodrugs can be bioconverted in a human liver to the active drug (Baba et al., The pharmacokinetics of enalapril in patients with compensated liver cirrhosis Br J Clin Pharmacol. 1990 June; 29(6):766-9). Thus, regardless of the mode of administration, human hepatocyte and blood biotransformation results may be used to evaluate ester prodrugs.

D-isoglutamyl-D-tryptophan or D-gamma-glutamyl-D-tryptophan (also known as H-D-Glu(D-Trp-OH)—OH or Apo805) is a synthetic hemoregulatory dipeptide developed for the treatment of autoimmune diseases including psoriasis (Sapuntsova, S. G., et al., Bulletin of Experimental Biology and Medicine, 2002, 133(5), 488-490). The sodium salt of H-D-Glu(D-Trp-OH)—OH (thymodepressin) is considered an effective treatment for psoriasis (U.S. Pat. No. 5,736,519), and is available as an injection ampoule in Russia.

### SUMMARY

This invention is based, at least in part, on the elucidation that a compound of Formula Q2:

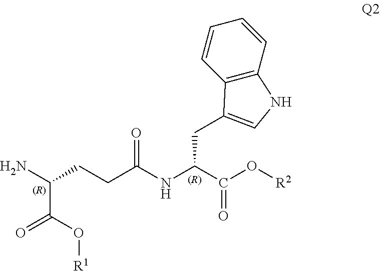

or a pharmaceutically acceptable salt thereof, wherein R¹ is selected from the group consisting of: C₁ to C₄ alkyl and benzyl; and R² is selected from the group consisting of: C₁ to C₄ alkyl and benzyl may be useful as a prodrug of thymodepressin.

Illustrative embodiments of the present invention provide a compound of Formula Q1:

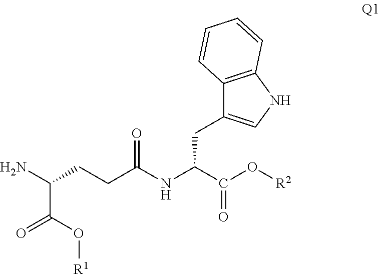

or a pharmaceutically acceptable salt thereof, wherein R¹ and R² are independently methyl, ethyl, isopropyl, or tert-butyl; or R¹ is benzyl and R² is methyl, ethyl, or benzyl.

Illustrative embodiments of the present invention provide a compound described herein wherein if R¹ is benzyl or methyl, then R² is ethyl, isopropyl or tert-butyl.

Illustrative embodiments of the present invention provide a compound described herein wherein R¹ is the same as R².

Illustrative embodiments of the present invention provide a compound described herein wherein R¹ and R² are methyl.

Illustrative embodiments of the present invention provide a compound described herein wherein R¹ and R² are ethyl.

Illustrative embodiments of the present invention provide a compound described herein wherein R¹ and R² are isopropyl.

Illustrative embodiments of the present invention provide a compound described herein wherein R¹ and R² are benzyl.

Illustrative embodiments of the present invention provide a compound described herein wherein R¹ and R² are tert-butyl.

Illustrative embodiments of the present invention provide a compound described herein wherein R¹ is ethyl, and R² is methyl.

Illustrative embodiments of the present invention provide a compound described herein wherein R¹ is ethyl, and R² is isopropyl.

Illustrative embodiments of the present invention provide a compound described herein wherein R¹ is tert-butyl, and R² is isopropyl.

Illustrative embodiments of the present invention provide a compound described herein wherein R¹ is benzyl, and R² is ethyl.

Illustrative embodiments of the present invention provide a pharmaceutical formulation comprising an effective amount of a compound of Formula Q1:

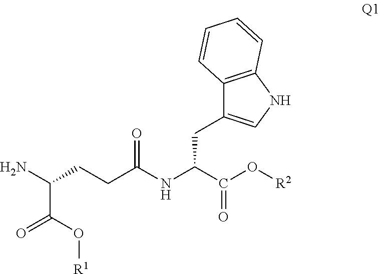

or a pharmaceutically acceptable salt thereof, wherein R¹ and R² are independently methyl, ethyl, isopropyl, or tert-butyl; or R¹ is benzyl and R² is methyl, ethyl, or benzyl.

Illustrative embodiments of the present invention provide a pharmaceutical composition described herein wherein R¹ is the same as R².

Illustrative embodiments of the present invention provide a pharmaceutical composition described herein wherein R¹ and R² are methyl.

Illustrative embodiments of the present invention provide a pharmaceutical composition described herein wherein R¹ and R² are ethyl.

Illustrative embodiments of the present invention provide a pharmaceutical composition described herein wherein R¹ and R² are isopropyl.

Illustrative embodiments of the present invention provide a pharmaceutical composition described herein wherein R¹ and R² are benzyl.

Illustrative embodiments of the present invention provide a pharmaceutical composition described herein wherein R¹ and R² are tert-butyl.

Illustrative embodiments of the present invention provide a pharmaceutical composition described herein wherein R¹ is ethyl, and R² is methyl.

Illustrative embodiments of the present invention provide a pharmaceutical composition described herein wherein R¹ is ethyl, and R² is isopropyl.

Illustrative embodiments of the present invention provide a pharmaceutical composition described herein wherein R¹ is tert-butyl, and R² is isopropyl.

Illustrative embodiments of the present invention provide a pharmaceutical composition described herein wherein R¹ is benzyl, and R² is ethyl.

Illustrative embodiments of the present invention provide a method of medical treatment of a subject at risk for or having psoriasis, the method comprising administering to the subject a therapeutically effective amount of a compound of Formula Q2:

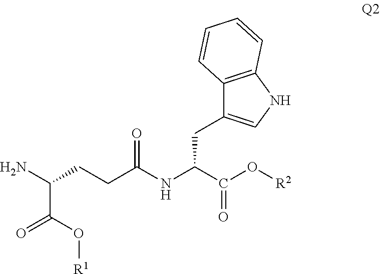

or a pharmaceutically acceptable salt thereof, wherein R¹ is selected from the group consisting of: C₁ to C₄ alkyl and benzyl; and R² is selected from the group consisting of: C₁ to C₄ alkyl and benzyl.

Illustrative embodiments of the present invention provide a method described herein wherein R¹ and R² are independently methyl, ethyl, isopropyl, or benzyl.

Illustrative embodiments of the present invention provide a method described herein wherein R¹ is the same as R².

Illustrative embodiments of the present invention provide a method described herein wherein R¹ and R² are methyl.

Illustrative embodiments of the present invention provide a method described herein wherein R¹ and R² are ethyl.

Illustrative embodiments of the present invention provide a method described herein wherein R¹ and R² are isopropyl.

Illustrative embodiments of the present invention provide a method described herein wherein R¹ and R² are benzyl.

Illustrative embodiments of the present invention provide a method described herein wherein R¹ is ethyl, and R² is methyl.

Illustrative embodiments of the present invention provide a method described herein wherein R¹ is ethyl, and R² is isopropyl.

Illustrative embodiments of the present invention provide a method described herein wherein R¹ is benzyl, and R² is ethyl.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

FIG. 1 shows the average (n=5) concentration of Apo805 in plasma after oral dosing of Apo804 or Apo805K1 (5 mg/kg) to rats demonstrating enhanced bioavailability of the pro-drug. Apo805K1 is the potassium salt of H-D-Glu(D-Trp-OH)—OH. Apo804 is the dipeptide H-D-Glu(D-Trp-OCH₃)—OCH₂Ph hydrochloride.

FIG. 2 shows the average (n=5) concentration of Apo805 in plasma after oral dosing of Apo838 or Apo805K1 (5 mg/kg) to rats demonstrating enhanced bioavailability of the pro-drug. Apo805K1 is the potassium salt of H-D-Glu(D-Trp-OH)—OH. Apo838 is the dipeptide H-D-Glu(D-Trp-OCH₂CH₃)—OCH₂CH₃ hydrochloride.

### DETAILED DESCRIPTION

As used herein, the term “alkyl” means a branched or unbranched saturated hydrocarbon chain. Non-limiting, illustrative examples of alkyl moieties include, methyl, ethyl, propyl, isopropyl, n-propyl, butyl, sec-butyl, isobutyl, n-pentyl, hexyl, octyl and the like. When the terminology “Cₓ-C_{y}”, where x and y are integers, is used with respect to alkyl moieties, the ‘C’ relates to the number of carbon atoms the alkyl moiety. For example, methyl may be described as a C₁ alkyl and isobutyl may be described as a C₄ alkyl. All specific integers and ranges of integers within each range are specifically disclosed by the broad range. For example, C₁-C₄, specifically includes the following: C₁, C₂, C₃, C₄, C₁-C₂, C₁-C₃, C₁-C₄, C₂-C₃, C₂-C₄, and C₃-C₄.

As used herein, the term “Boc-D-Glu(OH)—OR¹” refers to the structure:

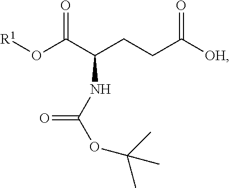

when R¹ is benzyl, it is the chemical 2-tert-butoxycarbonylamino-D-glutamic acid alpha-benzyl ester.

As used herein, the term “D-Trp-OR²” refers to the structure:

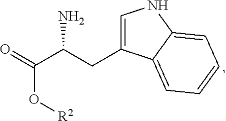

when R² is methyl, the compound is D-tryptophan methyl ester.

As used herein, the term “H-D-Glu(D-Trp-OH)—OH” or “H-D-iGlu-D-Trp-OH” or “gamma-D-glutamyl-D-tryptophan” refers to the structure:

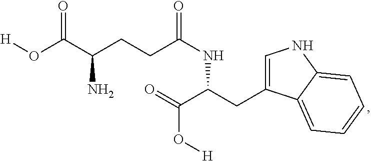

which is the drug thymodepressin.

As used herein, the term “Boc-D-Glu-(gamma-D-Trp-OR²)-alpha-OR¹” refers to the structure:

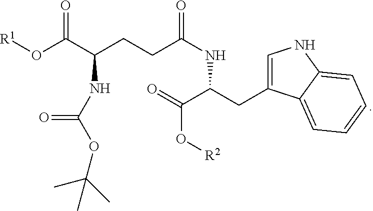

As used herein, the term “H-D-Glu-(gamma-D-Trp-OR²)-alpha-OR¹” refers to the structure:

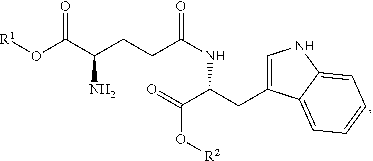

when R¹ is benzyl, R² is methyl, the compound is:

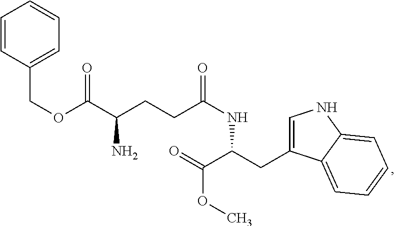

and the compound may be named as benzyl (2R)-2-amino-5-{[(2R)-3-(1H-indol-3-yl)-1-methoxy-1-oxopropan-2-yl]amino}-5-oxopentanoate.

As used herein, the term “H-D-Glu-(gamma-D-Trp-OR²)-alpha-OR¹” refers to the structure:

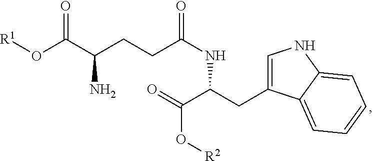

when R¹ is methyl, R² is methyl, the compound is

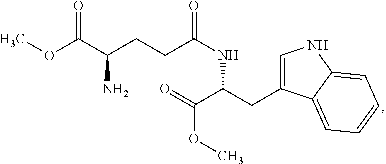

and the compound may be named as methyl (2R)-2-amino-5-{[(2R)-3-(1H-indol-3-yl)-1-methoxy-1-oxopropan-2-yl]amino}-5-oxopentanoate or gamma-D-glutamyl-D-tryptophan dimethyl ester.

As used herein, the term “H-D-Glu-(gamma-D-Trp-OR²)-alpha-OR¹” or H-D-Glu(D-Trp-OR²)—OR¹ refers to the structure:

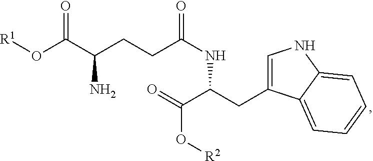

when R¹ is ethyl, R² is ethyl, the compound is

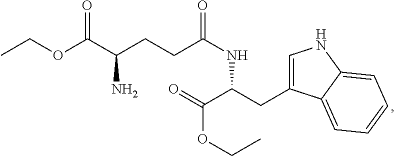

and the compound may be named as ethyl (2R)-2-amino-5-{[(2R)-3-(1H-indol-3-yl)-1-ethoxy-1-oxopropan-2-yl]amino}-5-oxopentanoate or gamma-D-glutamyl-D-tryptophan diethyl ester.

An acid addition salt is a salt formed after reacting the amine of H-D-iGlu-D-Trp-OH with inorganic acids including hydrochloric acid, sulphuric acid, hydrobromic acid, phosphoric acid, etc., or organic acids including formic acid, acetic acid, propionic acid, glycolic acid, lactic acid, pyruvic acid, oxalic acid, succinic acid, malic acid, tartaric acid, citric acid, trifluoroacetic acid, benzoic acid, salicylic acid, benzenesulphonic acid, and toluenesulphonic acids. It can also be formed from the acid deprotection of the Boc-D-iGlu-D-Trp-OH derivative.

A base addition salt is a salt formed from reacting the carboxylic acid of H-D-iGlu-D-Trp-OH with inorganic bases including sodium hydroxide, lithium hydroxide, potassium hydroxide, etc.

A process for the manufacture of H-D-iGlu-D-Trp-OH and its ammonium salt, free of inorganic salts, from the acid addition salts of H-D-iGlu-D-Trp-OH, which may be prepared from the dipeptide Boc-D-iGlu-D-Trp-OH is provided. Boc-D-iGlu-D-Trp-OH may be prepared from Boc-D-Glu(OH)—OR¹ and D-Trp-OR² wherein R¹ is selected from the group consisting of benzyl and C₁-C₄ alkyl and R² is C₁-C₄ alkyl with the proviso that C₄ alkyl is not tert-butyl.

Also provided is a process for the manufacture of H-D-iGlu-D-Trp-OH from a solution of the base addition salt of H-D-iGlu-D-Trp-OH, which is preferably prepared from the acid addition salt of the dipeptide H-D-Glu-(gamma-D-Trp-OR²)-alpha-OR¹ wherein each of R¹ and R² is independently selected from the group consisting of benzyl and C₁-C₄ alkyl.

Also provided is a method of using compounds described herein as a prodrug for thymodepressin in a subject, the method comprising administering to the subject a therapeutically effective amount of H-D-Glu-(gamma-D-Trp-OR²)— alpha-OR¹ wherein R¹, R² is independently C₁-C₄ alkyl, benzyl.

Provided is an aqueous phase process for the preparation of H-D-iGlu-D-Trp-OH, free of inorganic salts, which comprises:

(a) preparing a solution of H-D-iGlu-D-Trp-OH acid addition salt in an aqueous medium essentially free of organic solvent; or preparing a solution of H-D-iGlu-D-Trp-OH base addition salt in an aqueous medium essentially free of organic solvent;

(b) adjusting the pH to the predominant pH for the diacid form with an alkali metal hydroxide solution or a mineral acid, to cause the precipitation of H-D-iGlu-D-Trp-OH;

(c) recovering the precipitated H-D-iGlu-D-Trp-OH thereof; and

(d) vacuum drying the product resulting from step (c) to give H-D-iGlu-D-Trp-OH.

Also provided is a method for preparing the mono ammonium salt of H-D-iGlu-D-Trp-OH, free from inorganic salts, which method comprises the following steps:

(a) preparing a solution of H-D-iGlu-D-Trp-OH acid addition salt in an aqueous medium essentially free of organic solvent;

(b) adjusting the pH to the predominant pH for monovalent salt form with a metal hydroxide solution;

(c) subjecting the solution from step (b) to an ion-exchange resin and elution with water to exchange the metal ion from the salt in the solution for hydrogen ion until the eluant is at a pH of about 5.7 to about 7.0;

(d) contacting the ion-exchange resin with an ammonia based regenerant solution operative to exchange ions therein for the H-D-iGlu-D-Trp-OH of interest contained in the ion-exchange resin, thereby to form a regenerant eluant containing the ammonium salt of H-D-iGlu-D-Trp-OH;

(e) solvent evaporation of the solution from step (d) to give the crude ammonium salt;

(f) dissolving the ammonium salt from step (e) in water and slowly adding isopropanol so that a precipitate of the mono ammonium salt is formed; and

(g) vacuum drying the product from step (f) to give the crystalline form of H-D-iGlu-D-Trp-OH, ammonium salt (1:1).

Alternatively, in place of steps (f) and (g), the method may comprise the following steps:

(h) subjecting the material from step (e) to silica gel chromatography with isopropanol and ammonia solution as the eluant; and

(i) freeze-drying the product from step (h) to give the amorphous form of H-D-iGlu-D-Trp-OH, ammonium salt (1:1).

Also provided is a process for the preparation of the mono ammonium salt of H-D-iGlu-D-Trp-OH from crystalline H-D-iGlu-D-Trp-OH, free from inorganic salts, which process comprises the following steps:

(a) adding H-D-iGlu-D-Trp-OH to less than one equivalent of ammonium hydroxide solution;

(b) adjustment of the pH to 6 to 7 with ammonium hydroxide;

(c) solvent evaporation to give an oil; addition of isopropanol with stirring to cause the precipitation of the mono ammonium salt;

(d) recovering the precipitated H-D-iGlu-D-Trp-OH ammonium salt thereof; and

(e) vacuum drying the product resulting from step (c) to give H-D-iGlu-D-Trp-OH mono ammonium salt.

Also provided is a process for the preparation of an acid addition salt of D-isoglutamyl-D-tryptophan, wherein the salt is H-D-iGlu-D-Trp-OH hydrochloride, which process comprises:

(i) the base hydrolysis of a compound of Formula I:

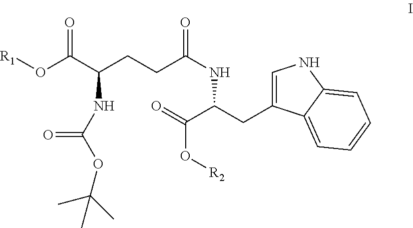

wherein R¹ is selected from the group consisting of C₁-C₄ alkyl and benzyl, and R² is C₁-C₄ alkyl with the proviso that C₄ alkyl is not tert-butyl,

with metal hydroxide in water and an inert solvent in the presence of methanol to give Boc-D-iGlu-D-Trp-OH, free from other diastereomers;

(ii) hydrogen chloride deprotection of Boc-D-iGlu-D-Trp-OH from step (i) in an inert organic solvent; and solvent evaporation to give the hydrochloride salt of H-D-iGlu-D-Trp-OH.

Also provided is a process for the preparation of a solution of the acid addition salt H-D-iGlu-D-Trp-OH hydrochloride, wherein the process comprises:

(a) the hydrogenation of a compound of formula II:

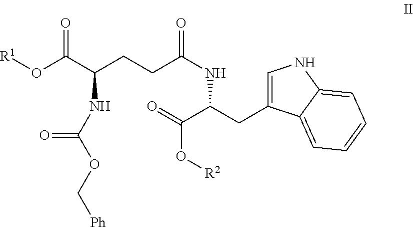

wherein R¹ is benzyl and R² is selected from the group consisting of benzyl and hydrogen, with palladium on charcoal in methanol or ethanol;

(b) purification of the crude H-D-iGlu-D-Trp-OH from step (a) with silica gel chromatography using isopropanol and water as an eluant; and

(c) treatment of the material from step (b) with hydrochloric acid in water to give a solution of the H-D-iGlu-D-Trp-OH hydrochloride salt in water.

Also provided is a process for the preparation of a solution of the base addition salt of H-D-iGlu-D-Trp-OH, wherein the process comprises:

(a) acid deprotection of the dipeptide N-Boc-D-Glu-(gamma-D-Trp-OR²)-alpha-OR¹, wherein each of R¹ and R² is independently selected from the group consisting of C₁-C₄ alkyl and benzyl;

(b) base hydrolysis of the product from step (a) with a metal hydroxide in water and an inert solvent in the presence of methanol wherein the metal hydroxide is selected from the group consisting of sodium hydroxide, potassium hydroxide and lithium hydroxide;

(c) extraction of the material from step (b) with a water immiscible solvent and separating the aqueous layer;

(d) adjusting the pH of the aqueous phase from step (c) from about 6 to about 7; and

(e) solvent evaporation of the solution from step (d) to produce a solution containing an estimated ratio of about a part solute to less than about 8 parts water wherein the solute is the base addition salt of D-isoglutamyl-D-tryptophan.

In another embodiment of the present invention, there is provided a novel diester derivative H-D-Glu-(gamma-D-Trp-OR²)-alpha-OR¹ hydrochloride wherein each of R¹ and R² is independently selected from the group consisting of benzyl and C₁-C₄ alkyl. These compounds can be used as intermediates for the preparation of the dipeptide D-isoglutamyl-D-tryptophan. Alternatively, H-D-Glu-(gamma-D-Trp-OR²)-alpha-OR¹ hydrochloride may be used in a pharmaceutical preparation wherein the hydrolysis of the ester takes place in situ to give D-isoglutamyl-D-tryptophan when the formulation is prepared. Alternatively these compounds may be used prodrugs for thymodepressin.

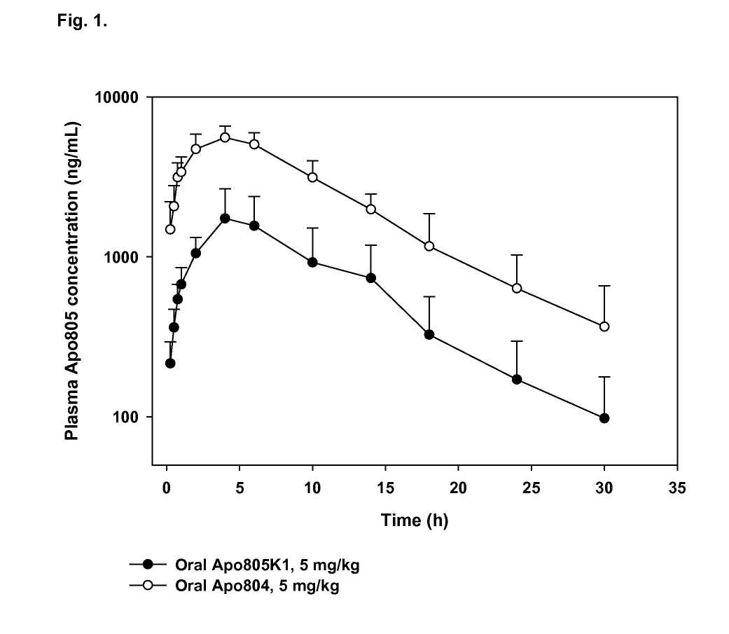

In another embodiment of the present invention, there is provided a pharmacokinetic study of a representative compound of formula H-D-Glu-(gamma-D-Trp-OR²)-alpha-OR¹ wherein R¹=benzyl, R²=methyl, in rats, as illustrated in FIG. 1.

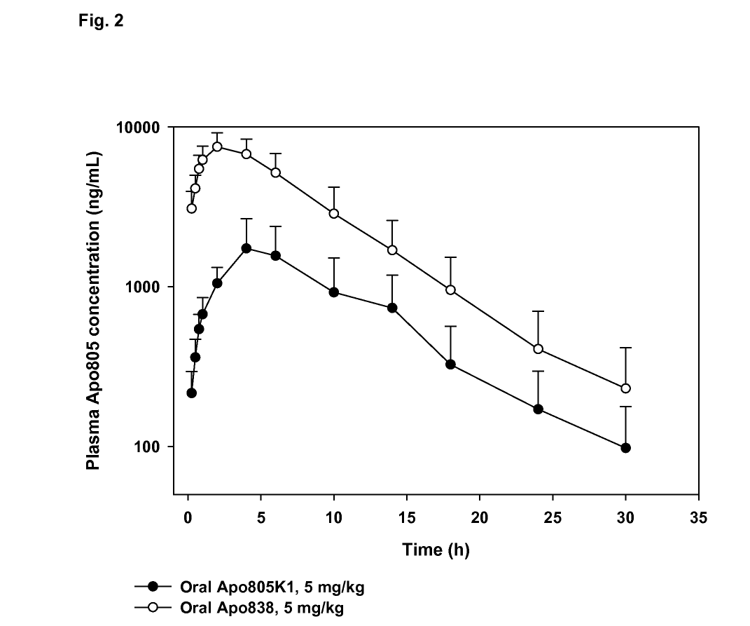

In another embodiment of the present invention, there is provided a pharmacokinetic study of a representative compound of formula H-D-Glu-(gamma-D-Trp-OR²)-alpha-OR¹ wherein R¹═R²=ethyl, in rats, as illustrated in FIG. 2.

Particular prodrug compounds of this invention are compounds having a structure of the formula H-D-Glu-(gamma-D-Trp-OR²)-alpha-OR¹:

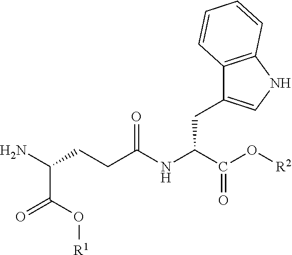

wherein

R¹═R²=methyl (Apo840); the compound is methyl (2R)-2-amino-5-{[(2R)-3-(1H-indol-3-yl)-1-methoxy-1-oxopropan-2-yl]amino}-5-oxopentanoate or gamma-D-glutamyl-D-tryptophan dimethyl ester or D-gamma-glutamyl-D-tryptophan dimethyl ester or H-D-Glu(D-Trp-OMe)-OMe;

R¹═R²=ethyl (Apo838); the compound is ethyl (2R)-2-amino-5-{[(2R)-1-ethoxy-3-(1H-indol-3-yl)-1-oxopropan-2-yl]amino}-5-oxopentanoate or gamma-D-glutamyl-D-tryptophan diethyl ester or D-gamma-glutamyl-D-tryptophan diethyl ester or H-D-Glu(D-Trp-OEt)-OEt;

R¹═R²=isopropyl (Apo845); the compound is propan-2-yl (2R)-2-amino-5-{[(2R)-3-(1H-indol-3-yl)-1-oxo-1-(propan-2-yloxy)propan-2-yl]amino}-5-oxopentanoate or gamma-D-glutamyl-D-tryptophan diisopropyl ester or D-gamma-glutamyl-D-tryptophan diisopropyl ester or H-D-Glu(D-Trp-O-isopropyl)-O-isopropyl;

R¹=benzyl and R²=methyl (Apo804); the compound is benzyl (2R)-2-amino-5-{[(2R)-3-(1H-indol-3-yl)-1-methoxy-1-oxopropan-2-yl]amino}-5-oxopentanoate or H-D-Glu(D-Trp-O-methyl)-O-benzyl;

R¹=ethyl, R²=methyl (Apo916); the compound is ethyl (2R)-2-amino-5-{[(2R)-3-(1H-indol-3-yl)-1-methoxy-1-oxopropan-2-yl]amino}-5-oxopentanoate or H-D-Glu(D-Trp-O-Me)-O-Et.;

R¹=ethyl, R²=isopropyl (Apo915); the compound is ethyl (2R)-2-amino-5-{[(2R)-3-(1H-indol-3-yl)-1-oxo-1-(propan-2-yloxy)propan-2-yl]amino}-5-oxopentanoate hydrochloride or H-D-Glu(D-Trp-O-isopropyl)-O-Et.HCl;

R¹=tert-butyl, R²=tert-butyl (Apo920); the compound is tert-butyl (2R)-2-amino-5-{[(2R)-1-tert-butoxy-3-(1H-indol-3-yl)-1-oxopropan-2-yl]amino}-5-oxopentanoate or H-D-Glu(D-Trp-O-t-Bu)-O-t-Bu;

R¹=tert-butyl, R²=isopropyl (Apo919); the compound is tert-butyl (2R)-2-amino-5-{[(2R)-3-(1H-indol-3-yl)-1-oxo-1-(propan-2-yloxy)propan-2-yl]amino}-5-oxopentanoate hydrochloride or H-D-Glu(D-Trp-O-t-Bu)-O-isopropyl;

R¹=benzyl, R²=ethyl (Apo925); the compound is benzyl (2R)-2-amino-5-{[(2R)-1-ethoxy-3-(1H-indol-3-yl)-1-oxopropan-2-yl]amino}-5-oxopentanoate hydrochloride or H-D-Glu(D-Trp-O-Et)-O-Bzl;

R¹═R²=benzyl (Apo926); the compound is benzyl (2R)-2-amino-5-{[(2R)-1-(benzyloxy)-3-(1H-indol-3-yl)-1-oxopropan-2-yl]amino}-5-oxopentanoate or H-D-Glu(D-Trp-O-Bzl)-O-Bzl;

The present invention provides, as depicted below in Scheme 5, a reliable methodology for the high yield synthesis of pure N-(tert-butoxycarbonyl)-D-isoglutamyl-D-tryptophan.

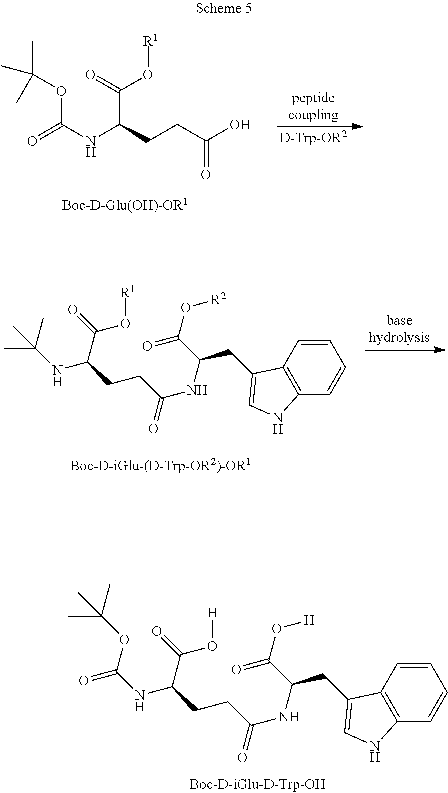

A solution of Boc-D-Glu(OH)—OR¹ wherein R¹ is benzyl in an inert solvent is reacted with N-(3-dimethylaminopropyl)-N′-ethylcarbodiimide hydrochloride (EDC), hydroxybenzotriazole (HOBt) and diisopropylethylamine (DIPEA). The preferred temperature is from about 5 to about −5° C. and the preferred solvent is dichloromethane. After mixing for about 5 to about 30 minutes, preferably about 15 minutes, a solution of the HCl salt of D-Trp-OR² wherein R² is methyl with diisopropylethylamine (DIPEA) is added dropwise. The resulting solution is stirred at the ice-cold temperature, preferably from about −5 to about 5° C. for about 1 hour and then at room temperature for about 12 to about 20 hrs, preferably about 16 hrs. The product Boc-D-iGlu-(D-Trp-OR²)-alpha-OR¹ is isolated by conventional means. The compound can be easily crystallized from ethyl acetate and hexanes. The two synthetic impurities present in trace amounts are compounds (A) and (B), which can be removed by recrystallization. Both compounds are believed to derive from the reagent HOBt.

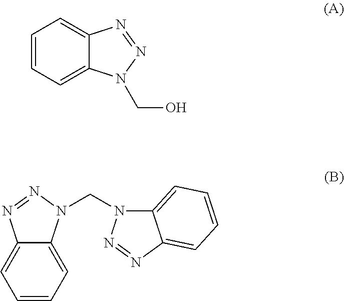

The diester Boc-D-Glu-(gamma-D-Trp-OR²)-alpha-OR¹ in alcohol is mixed with a solution of sodium hydroxide. The preferred amount of sodium hydroxide is about 2.5 to about 5 equivalents per equivalent of the diester. Still preferred, a molar ratio of about 2 to about 3.5 moles of NaOH per mole of the diester compound is used. The preferred ratio of solvent is about 2 mL of alcohol per mL of water, and the preferred ratio of NaOH to water is about 1 g to 20 mL. This isolation procedure involves the extraction of the reaction mixture with ethyl acetate, thus any organic impurity is removed at this stage of the synthesis. Upon acidification, the aqueous fraction is extracted with an organic solvent such as ethyl acetate. The diacid N-(tert-butoxycarbonyl)-D-isoglutamyl-D-tryptophan (Boc-D-iGlu-D-Trp-OH) is isolated by conventional means as a solid. The isolated yield for the combined two steps is typically 89% yield. The processes of the present invention are further illustrated in the examples below.

Detailed investigation and monitoring of the hydrolysis step showed that the compound Boc-D-iGlu-(D-Trp-OR²)—OR¹ wherein R¹ is benzyl and R² is methyl is first converted by methanol to give Boc-D-iGlu-(D-Trp-OMe)-OMe, and then the compound is hydrolyzed into the diacid. Methanol is a beneficial for the rapid hydrolysis of the alpha benzyl ester. In view of the large volume of ethyl acetate required for extraction of the product, a two phase procedure for the efficient hydrolysis of Boc-D-iGlu-(D-Trp-OR²)—OR¹ is disclosed. A mixture of Boc-D-iGlu-(D-Trp-OR²)—OR¹ in tert-butyl methyl ether (MTBE) and a metal hydroxide solution such as lithium hydroxide or sodium hydroxide solution is stirred. The preferred ratio of metal hydroxide to Boc-D-iGlu-(D-Trp-OR²)—OR¹ is between about 2.0-2.5 to 1. Methanol is added and the mixture is vigorously stirred for about 1 to about 6 hrs, preferably from about 1.5 to about 2.5 hours. The mixture is isolated from the organic phase by conventional means. This procedure eliminates the use of large amount of ethyl acetate for extraction and is illustrated in the examples below.

In a conventional method, the compound Boc-D-iGlu-D-Trp-OH is deprotected with organic acids to give the dipeptide H-D-iGlu-D-Trp-OH which requires extensive purification. There are numerous shortcomings to using formic acid at 40° C. to deprotect a mixture of Boc-D-iGlu-D-Trp-OH and Boc-D-Glu-D-Trp-OH to give a mixture of H-D-iGlu-D-Trp-OH and H-D-Glu-D-Trp-OH. Ion exchange chromatography and reverse HPLC are used to isolate the product in such a process. The recovery is low and the procedure is not amenable to large scale production using such a method. The deprotection of N-tert-butoxycarbonyl group with trifluoroacetic acid or formic acid generates tert-butyl carbonium ion which may react with the indole nitrogen to form the N-tert-butyl product (Löw, M., et. al. (1978), Hoppe-Seyler's Z. Physiol. Chem., 359(12):1643-51). The formation of the glutarimide (1.8) (Pandit, U.K. (1989), Pure & Appl. Chem., Vol. 61, No. 3, pp. 423-426) is another concern.

The acid addition salt of the present invention, in particular the crude hydrochloride salt, can be easily prepared with HCl in an inert solvent such as ethyl acetate at low temperature, preferably from about 0° C. to about ambient temperature. Solvent evaporation affords the HCl salt of H-D-Glu(D-Trp-OH)—OH which may be used towards the preparation of H-D-Glu(D-Trp-OH)—OH.

In determining the pH for the precipitation of H-D-Glu(D-Trp-OH)—OH in its diacid form, a theoretical calculation on a speciation plot shows that H-D-Glu(D-Trp-OH)—OH exists in the diacid form at pH of about 2.5 to about 3.0.

The deprotection of Boc-H-D-iGlu-D-Trp-OH with trifluoroacetic acid in an inert solvent affords the trifluoroacetic acid salt. The inert solvent is dichloromethane and usually a 1:1 mixture of trifluoroacetic acid and dichloromethane is used. Solvent evaporation affords an oil which is vacuum dried to remove residual solvent. The oil is dispersed in water. When the pH is adjusted to about 3.0, a white solid appears after stirring for about 12 to about 16 hrs.

In the preparation of the acid addition salt, it is preferable to use HCl in an inert solvent to produce the hydrochloride salt. Alternatively, the trifluoroacetic acid salt can be produced using the method above. The use of the hydrochloric acid salt as the acid addition salt is preferred because the deprotection of Boc-D-iGlu-D-Trp-OH is more efficient with HCl in an inert solvent such as 3M HCl in ethyl acetate. The reaction time is significantly longer with trifluoroacetic acid. In addition, the trifluoroacetic acid salt of D-iGlu-D-Trp-OH contains several synthetic impurities, which carry over to the D-iGlu-D-Trp-OH upon precipitation at pH of about 2.5 to about 3.0 in water. The impurities must be removed by extensive recrystallization.

The starting material Boc-D-iGlu-D-Trp-OH is prepared using the methodology as described earlier. The acid addition salt should be vacuum dried to ensure it is free from organic solvent and volatile impurities. In the precipitation of thymodepressin in water, a solution of the acid addition in water is prepared. The ratio of acid addition salt to water is in the range of about 1:5 to about 1:10. Still preferred, the ratio of acid addition salt is in the range of about 1:6 to about 1:8. A metal hydroxide solution, normally a sodium hydroxide solution is used to precipitate the product, but potassium hydroxide and other metal hydroxide solutions can be used.

Crude H-D-Glu(D-Trp-OH)—OH (also known as H-D-iGlu-D-Trp-OH) can also be prepared by the hydrogenation of a compound of formula II:

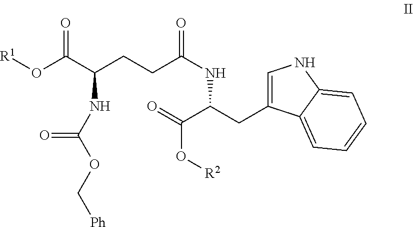

wherein R¹ is benzyl and R² is selected from the group consisting of benzyl and hydrogen, with palladium on charcoal in methanol or ethanol. After filtration of the catalyst, the filtrate is evaporated to an oil, which is further purified with silica gel chromatography using isopropanol and water as an eluant. The H-D-Glu(D-Trp-OH)—OH obtained can be converted to the H-D-Glu(D-Trp-OH)—OH hydrochloride salt in water with hydrochloric acid.

A solution of the base addition salt of D-isoglutamyl-D-tryptophan is prepared by the acid deprotection of the dipeptide Boc-D-Glu-(gamma-D-Trp-OR²)-alpha-OR¹, wherein each of R¹ and R² is independently selected from the group consisting of benzyl and C₁-C₄ alkyl. For example, HCl deprotection of Boc-D-Glu-(gamma-D-Trp-OR²)-alpha-OR¹ in an inert solvent such as dichloromethane, affords the HCl salt of H-D-Glu-(gamma-D-Trp-OR²)-alpha-OR¹. For the combination where R¹ is benzyl and R² is methyl, the product HCl.H-D-Glu-(gamma-D-Trp-OR²)-alpha-OR¹ precipitates out of dichloromethane and can be removed by filtration. Treatment of the acid addition salt with a metal hydroxide in an inert solvent such as methanol for one phase homogeneous hydrolysis or with tert-butyl methyl ether for two phase hydrolysis affords H-D-Glu(D-Trp-OH)—OH (also known as H-D-iGlu-D-Trp-OH) base addition salt in solution. Upon extraction of the reaction mixture with a water immiscible solvent such as ethyl acetate or tert-butyl methyl ether, the aqueous phase is neutralized to pH of about 6 to about 7 and the solution is evaporated to reduced the volume to an estimated ratio of less than about 1 part solute:8 parts water. The solute is the base addition salt (in mono carboxylate form) of H-D-Glu(D-Trp-OH)—OH. If sodium hydroxide is used as the metal hydroxide, the solute will be the mono sodium form of the H-D-Glu(D-Trp-OH)—OH in water. Adjustment of this solution to pH of about 2.5 to about 3.0 will result in the precipitation of solid H-D-Glu(D-Trp-OH)—OH.

The mono ammonium salt of H-D-Glu(D-Trp-OH)—OH can be prepared directly from the Boc-D-iGlu-D-Trp-OH dipeptide. The crude acid addition salt such as the hydrochloride salt, prepared in the manner as described above, is treated with ion exchange resin to remove the inorganic salt. Thus, a solution of the crude thymodepressin hydrochloride is dissolved in water and adjusted to pH of about 6 to about 8. The solution is treated with ion exchange resin. The preferred resin is a sulfonic acid based resin. An example of such is AMBERLYST® 15. The inorganic salt is removed by washing with water until pH of about 5.7 to about 7. Ammonia is used as a regenerant to recover the ammonium salt of thymodepressin from the resin. It is preferred to use concentrated ammonia and isopropanol as a regenerant. The preferred ratio is concentrated ammonia and isopropanol in the ratio of about 1 to about 3-4, with the final wash using concentrated ammonia and isopropanol in the ratio of 1 part concentrated ammonia:1 part water:2 part isopropanol. The ammonia wash is evaporated under reduced pressure to an oil, which is crystallized with isopropanol and water to give the mono ammonia salt as a white solid. The preferred ratio of isopropanol to water for recrystallization is in the range of about 5:1 to about 10:1. Column chromatography is not required.

Crude H-D-Glu(D-Trp-OH)—OH can also be purified to pharmaceutical grade purity by flash silica gel chromatography with isopropanol and water as an eluant. The preferred mobile phase is isopropanol:water in the range of about 10:1 to about 5:1. The product may be isolated by conventional means.

In a similar manner, the mono ammonium salt can also be purified by flash silica gel chromatography with isopropanol and concentrated ammonia as an eluant. The preferred mobile phase is isopropanol:ammonia in the range of about 10:1 to about 5:1. The product may be isolated by conventional means.

The D-isoglutamyl-D-tryptophan mono ammonium salt obtained from crystallization using isopropanol and water is crystalline. On the other hand, when a solution of D-isoglutamyl-D-tryptophan mono ammonium salt is freeze-dried, the amorphous material is obtained.

Extensive study was conducted to confirm that there is no racemization of the chiral centers in the column purification and throughout the reaction sequence, and the details are shown in the examples below.

According to the present invention, a method is provided for the synthesis of Boc-D-iGlu-D-Trp-OH, free from the alpha amide isomer. A method is provided to convert the Boc-D-iGlu-D-Trp-OH to the acid addition salt of thymodepressin, in particular the hydrochloride salt. Speciation plot prediction affords a method for the precipitation of H-D-Glu(D-Trp-OH)—OH in pure form at a pH of about 3 in water. In addition, a method is provided to clean up less than 97% pure H-D-Glu(D-Trp-OH)—OH by flash column chromatography using isopropanol and water as an eluant. Another aspect of this invention involves a convenient method for the preparation of the mono ammonium salt from the hydrochloride salt of H-D-Glu(D-Trp-OH)—OH. The inorganic salt is removed by ion exchange resin and the mono ammonium salt recovered by using an ammonia based regenerant solution. The mono ammonium salt can be obtained by crystallization in pure form. A method is also provided to purify mono ammonium salt of lower purity by flash silica gel column chromatography using isopropanol and water as an eluant.

The synthesis process intermediate, H-D-Glu-(gamma-D-Trp-OR²)-alpha-OR¹ wherein R¹ and R² are independently C₁-C₄ alkyl, benzyl; is also a prodrug of thymodepressin. The hydrolysis of the diester takes place in-vivo to release the parent drug H-D-Glu(D-Trp-OH)—OH.

For example, the pharmacokinetic study of representative compounds of formula H-D-Glu-(gamma-D-Trp-OR²)-alpha-OR¹ in rats is shown in FIGS. 1 and 2 below. The compound shows significantly better oral exposure than the parent drug Apo805K1 (the potassium salt of H-D-Glu(D-Trp-OH)—OH) in the pharmacokinetic study.

The sodium salt of H-D-Glu(D-Trp-OH)—OH has been used for the treatment of psoriasis and is only available in an injection ampoule in Russia. The prodrug H-D-Glu-(gamma-D-Trp-OR²)-alpha-OR¹ wherein R¹ and R² are as defined above, may be formulated into pharmaceutical compositions for administration to subjects in a therapeutically active amount and in a biologically compatible form suitable for in vivo administration, i.e., a form of the peptides to be administered in which any toxic effects are outweighed by the therapeutic effects.

According to the pharmacokinetic studies as shown in FIGS. 1 and 2, the prodrug H-D-Glu-(gamma-D-Trp-OR²)-alpha-OR¹ is effectively biotransformed into H-D-Glu(D-Trp-OH)—OH (Apo805) in rats after oral administration with significant improvement in oral exposure than Apo805K1. In human hepatocyte studies, the prodrug H-D-Glu-(gamma-D-Trp-OR²)-alpha-OR¹ is also biotransformed in human hepatocytes into Apo805. The level of biotransformation is in-line with the observation of other prodrug transformation such as the prodrug enalapril to the drug enalaprilate. For example, the compounds such as H-D-Glu-(gamma-D-Trp-OEt)-alpha-OEt hydrochloride, and H-D-Glu-(gamma-D-Trp-OMe)-alpha-OCH₂Ph hydrochloride are solids and are extremely water soluble. Therefore, it is also an excellent candidate for formulation,

Administration of the prodrug H-D-Glu-(gamma-D-Trp-OR²)-alpha-OR¹ and/or its pharmaceutically acceptable salt as described herein can be via any of the accepted modes of administration for systemically active therapeutic medicaments. These methods include oral, parenteral and otherwise systemic, aerosol or topical forms.

Depending on the intended mode of administration, the compositions used may be in the form of solid, semi-solid or liquid dosage forms, such as, for example, tablets, suppositories, pills, capsules, powders, liquids, aerosols, suspensions, or the like, preferably in unit dosage forms suitable for single administration of precise dosages. The compositions will include at least one conventional pharmaceutical carrier or excipient and the prodrug of formula H-D-Glu-(gamma-D-Trp-OR²)-alpha-OR¹ or its pharmaceutically acceptable salt and, in addition, may include other medicinal agents, pharmaceutical agents, carriers, adjuvants, etc.

For solid compositions, conventional non-toxic solid carriers including, for example, pharmaceutical grades of mannitol, lactose, starch, magnesium stearate, sodium saccharin, talcum, cellulose, glucose, sucrose, magnesium carbonate, and the like may be used. The active compound as defined above may be formulated as suppositories using, for example, polyalkylene glycols, for example, propylene glycol, as the carrier. Liquid pharmaceutically administerable compositions can, for example, be prepared by dissolving, dispersing, etc., the active compound as defined above and optional pharmaceutical adjuvants in a carrier, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; for example, see Remington: The Science and Practice of Pharmacy, David B. Troy (Ed.), Lipincott Williams & Wilkins, Philadelphia, Pa., 21^{st} Edition, 2006. The composition or formulation to be administered will, in any event, contain a quantity of the active compound(s) in an amount effective to alleviate the symptoms of the subject being treated.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, etc.

For the prodrug H-D-Glu-(gamma-D-Trp-OR²)-alpha-OR¹ or the mono ammonium salt of H-D-Glu(D-Trp-OH)—OH, either oral or nasal (bronchial) administration is preferred, depending on the nature of the disorder being treated.

For oral administration, a pharmaceutically acceptable non-toxic composition is formed by the incorporation of any of the normally employed excipients, such as, for example pharmaceutical grades of mannitol, lactose, starch, magnesium stearate, sodium saccharin, talcum, cellulose, glucose, sucrose, magnesium, carbonate, and the like. Such compositions take the form of solutions, suspensions, tablets, pills, capsules, powders, sustained release formulations and the like. Such compositions may contain from about 1% to about 95% active ingredient, preferably from about 25% to about 70%.

Oral and nasal administration to the lungs can also be effected by aerosol delivery forms. For aerosol administration, the active ingredient is preferably supplied in finely divided form along with a surfactant and a propellant. Typical percentages of active ingredients are from about 0.01 to about 20% by weight, preferably from about 0.04% to about 1.0%.

Surfactants must, of course, be non-toxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olestearic and oleic acids with an aliphatic polyhydric alcohol or its cyclic anhydride such as, for example, ethylene glycol, glycerol, erythritol, arabitol, mannitol, sorbitol, the hexitol anhydrides derived from sorbitol (the sorbitan esters sold under the name SPAN®) and the polyoxyethylene and polyoxypropylene derivatives of these esters. Mixed esters, such as mixed or natural glycerides may be employed. The preferred surface-active agents are the oleates or sorbitan, e.g., those sold under the names ARLACEL®C (Sorbitan sesquioleate), SPAN®80 (sorbitan monooleate) and SPAN®85 (sorbitan trioleate). The surfactant may constitute from about 0.1% to about 20% by weight of the composition, preferably from about 0.25% to about 5%.

The balance of the composition is ordinarily propellant. Liquefied propellants are typically gases at ambient conditions, and are condensed under pressure. Among suitable liquefied propellants are the lower alkanes containing up to five carbons, such as butane and propane; and preferably fluorinated or fluorochlorinated alkanes, such as are sold under the name FREON®. Mixtures of the above may also be employed.

In producing the aerosol, a container equipped with a suitable valve is filled with the appropriate propellant, containing the finely divided active ingredient and surfactant. The ingredients are thus maintained at an elevated pressure until released by action of the valve.

For topical administration, these compositions comprise an effective amount of a compound of this class in admixture with at least one pharmaceutically acceptable non-toxic carrier. A suitable range of composition would be from about 0.1% to about 10% active ingredient, and the balance being the carriers, preferably from about 1% to about 2% active ingredient. The concentration of active ingredient in pharmaceutical compositions suitable for topical application will vary depending upon the particular activity of the compound used in conjunction with the condition and subject to be treated. Suitable carriers or medicament vehicles for topical application of these compounds include creams, ointments, lotions, emulsions, solutions and the like.

For example, a suitable ointment for topical application of the compounds of the present invention contains from about 15 to about 45 percent of a saturated fatty alcohol, having 16 to 24 carbon atoms such as cetyl alcohol, stearyl alcohol, behenyl alcohol, and the like, and from about 45 to about 85 wt. percent of a glycol solvent such as propylene glycol, polyethylene glycol, dipropylene glycol, and mixtures thereof. The ointment can also contain from about 0 to about 15 wt. percent of a plasticizer such as polyethylene glycol, 1,2,6-hexanetriol, sorbitol, glycerol, and the like; from about 0 to about 15 wt. percent of a coupling agent such as a saturated fatty acid having from 16 to 24 carbon atoms, e.g., stearic acid, palmitic acid, behenic acid, a fatty acid amide e.g., oleamide, palmitamide, stearamide, behenamide and an ester of a fatty acid having from 16 to 24 carbon atoms such as sorbitol monostearate, polyethylene glycol monostearate, polypropylene glycol or the corresponding mono-ester of other fatty acids such as oleic acid and palmitic acid; and from about 0 to about 20 wt. percent of a penetrant such as dimethyl sulfoxide or dimethylacetamide.

A therapeutically active amount of the prodrug H-D-Glu-(gamma-D-Trp-OR²)-alpha-OR¹ or its pharmaceutically acceptable salt may vary according to factors such as disease state, age, sex, and weight of the individual. Dosage regime may be altered to provide the optimum therapeutic response. Generally, the daily regimen should be in the range of from about 1 to about 200 mg of peptide.

The following are examples of representative formulations and in no way restrict the scope of in the preparation of different pharmaceutical compositions:

| Ingredients | Quantity per tablet (mgs) |
|---|---|
| Active ingredient | 25 |
| lactose, spray-dried | 20 |
| corn starch | 153 |
| magnesium stearate | 2 |

The above ingredients are thoroughly mixed and pressed into single scored tablets.

| Ingredients | Quantity per tablet (mgs) |
|---|---|
| Active ingredient | 100 |
| lactose, spray-dried | 148 |
| magnesium stearate | 2 |

The above ingredients are mixed and introduced into a hard-shell gelatin capsule.

| Ingredients | Quantity per tablet (mgs) |
|---|---|
| Active ingredient | 200 |
| lactose | 145 |
| corn starch | 50 |
| magnesium stearate | 5 |

The above ingredients are mixed intimately and pressed into single scored tablets.

| Ingredients | Quantity per tablet (mgs) |
|---|---|
| Active ingredient | 108 |
| lactose | 15 |
| corn starch | 25 |
| magnesium stearate | 2 |

The above ingredients are mixed and introduced into a hard-shell gelatin capsule.

| Ingredients | Quantity per tablet (mgs) |
|---|---|
| Active ingredient | 150 |
| lactose | 92 |

The above ingredients are mixed and introduced into a hard-shell gelatin capsule.

An injectable preparation buffered to a pH of about 7 is prepared having the following composition:

| Ingredients | | |
|---|---|---|
| Active ingredient | 0.2 | g |
| KH₂PO₄ | 2 | ml |

| | |
|---|---|
| KOH (1N) | q.s. to pH 7 |
| Water (distilled, sterile) | q.s. to 20 ml |

An injectable preparation buffered to a pH of about 7 is prepared having the following composition:

| Ingredients | |
|---|---|
| Active ingredient | 0.01 g |
| Water (distilled, sterile) | q.s. to 1 ml |
| NaOH (0.2N) | q.s. to pH 7 |

An oral suspension is prepared having the following composition:

| Ingredients | | |
|---|---|---|
| Active ingredient | 0.1 | g |
| fumaric acid | 0.5 | g |
| methyl paraben | 2.0 | g |
| granulated sugar | 0.1 | g |
| sorbitol (70% solution) | 25.5 | g |
| VEEGUM ® K (Vanderbilt Co.) | 12.85 | g |
| Flavoring | 1.0 | g |
| Colorings | 0.035 | ml |

| | |
|---|---|
| distilled water | q.s. to 100 ml |

Topical Formulation

| Ingredients | Grams |
|---|---|
| Active compound | 0.2-2 |
| SPAN ® 60 | 2 |
| TWEEN ® 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| distilled water | q.s. 100 ml |

All of the above ingredients, except water, are combined and heated to about 45 degrees C. with stirring. A sufficient quantity of water at about 45 degrees C. is then added with vigorous stirring to emulsify the ingredients, and water then added q.s. 100 g.

A pharmacokinetic study of a representative compound of formula H-D-Glu-(gamma-D-Trp-OR²)-alpha-OR¹ wherein R¹═R²=ethyl, in rats, is shown in FIG. 2 below. The compound shows improved oral exposure than the parent drug Apo805K1 (potassium salt of H-D-Glu(D-Trp-OH)—OH) in the pharmacokinetic study.

Some compounds of the present invention or for use in this invention may be represented by the Formula Q2:

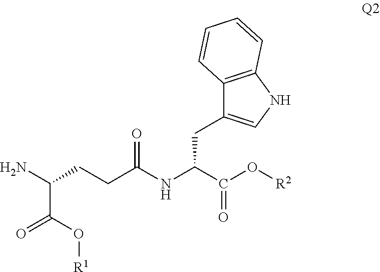

or a pharmaceutically acceptable salt thereof, wherein R¹ is selected from the group consisting of: C₁ to C₄ alkyl and benzyl; and R² is selected from the group consisting of: C₁ to C₄ alkyl and benzyl.

In some embodiments, the compound is a compound in which R¹ is methyl and R² is methyl.

In some embodiments, the compound is a compound in which R¹ is ethyl and R² is ethyl.

In some embodiments, the compound is a compound in which R¹ is isopropyl and R² is isopropyl.

In some embodiments, the compound is a compound in which R¹ is benzyl and R² is methyl.

In some embodiments, the compound is a compound in which R¹ is ethyl and R² is methyl.

In some embodiments, the compound is a compound in which R¹ is ethyl and R² is isopropyl.

In some embodiments, the compound is a compound in which R¹ is tert-butyl and R² is tert-butyl.

In some embodiments, the compound is a compound in which R¹ is tert-butyl and R² is isopropyl.

In some embodiments, the compound is a compound in which R¹ is benzyl and R² is ethyl.

In some embodiments, the compound is a compound in which R¹ is benzyl and R² is benzyl.

### General Processes for Preparation of a Compound of Formula Q2

Compounds of Formula Q1 wherein R¹ and R² are the same alkyl group may be prepared by the following Process A.

Process A may be used for the preparation of a compound of Formula Q2 wherein R¹═R².

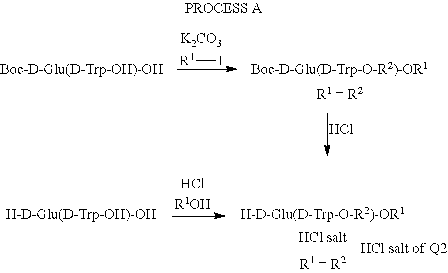

In process A, the dipeptide Boc-D-Glu(D-Trp-OH)—OH may be treated with potassium carbonate and R¹—I to give the diester Boc-D-Glu(D-Trp-O—R²)—O—R¹ wherein R¹ and R² are the same alkyl group. R¹—I is an primary alkyl iodide or secondary alkyl iodide. Deprotection of the Boc group with HCl in an inert solvent such as dioxane, or ethyl acetate affords the compound of Formula Q2 wherein R¹ and R² are the same alkyl. Alternatively, the compound of Formula Q2 wherein R¹ and R² are the same alkyl is prepared from the reaction of H-D-Glu(D-Trp-OH)—OH in the alcohol R¹⁰H in presence of HCl.

The method described in process A is further illustrated in example 17 below. Boc-D-Glu(D-Trp-OH)—OH is reacted with potassium carbonate and 2-2-iodopropane in DMF to give Boc-D-Glu(D-Trp-O—R²)—O—R¹ wherein R¹ and R² are isopropyl. The reagent R¹—I is 2-iodopropane. Deprotection of the Boc group with HCl in an inert solvent affords the compound H-D-Glu(D-Trp-O-isopropyl)-β-isopropyl.HCl.

In example 16, H-D-Glu(D-Trp-OH)—OH is reacted with HCl in R¹OH wherein R¹ is methyl to give the compound H-D-Glu(D-Trp-O—CH₃)—O—CH₃.HCl

Compounds of Formula Q2 wherein R¹ and R² are independently C₁-C₄ alkyl or benzyl may be prepared by Process B.

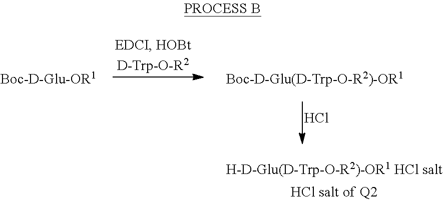

In Process B, the Boc-D-Glu-O—R¹ is coupled with D-Trp-O—R² in the presence of EDCl and HOBt to give the compound Boc-D-Glu-(D-Trp-O—R²)—O—R¹. HCl deprotection as described under process A affords the compound of Formula Q2.

The method described in process B is further illustrated in example 21 and 22. In example 22, Boc-D-Glu-O—R¹ wherein R¹ is benzyl is coupled with D-Trp-O—R² wherein R² is ethyl in the presence of EDCl and HOBt to give the compound Boc-D-Glu-(D-Trp-O—R²)—O—R¹ wherein R¹ is benzyl and R² is ethyl. The compound is isolated by traditional means. HCl deprotection of Boc-D-Glu-(D-Trp-O—R²)—O—R¹ in ether affords the compound of formula IA, H-D-Glu(D-Trp-O—R²)—O—R¹ wherein R¹ is benzyl and R² is ethyl. The compound in example 22 is H-D-Glu(D-Trp-O-Et)-O-Bzl.HCl.

Compounds of Formula Q2 wherein R¹ and R² are independently C₁-C₄ alkyl may be prepared by Process C.

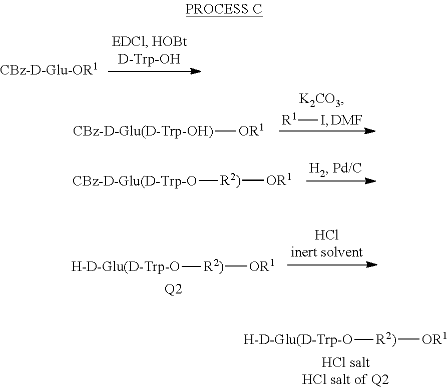

In Process C, the CBz-D-Glu-O—R¹ is coupled with D-Trp-OH in the presence of EDCl and HOSu in an inert solvent such as DMF to give the compound CBz-D-Glu(D-Trp-OH)—O—R¹. Treatment of CBz-D-Glu(D-Trp-OH)—O—R¹ with potassium carbonate in DMF and R²—I gives the compound CBz-D-Glu(D-Trp-O—R²)—O—R¹. R²—I is a primary alkyl or secondary alkyl iodide. Hydrogenation of a solution of CBz-D-Glu(D-Trp-O—R²)—O—R¹ in ethanol over Pd/C affords the compound of Formula Q2. The HCl salt of a compound of formula Q2 is prepared by reacting the compound of formula Q2 with HCl in an inert solvent such as dioxane or ether or ethyl acetate. The method described in process C is further illustrated in example 18 and 19.

In example 18, CBz-D-Glu-O—R¹ wherein R¹ is ethyl, is coupled with D-Trp-OH in the presence of EDCl and HOSu in an inert solvent such as DMF to give the compound CBz-D-Glu(D-Trp-OH)—O-Et. Treatment of CBz-D-Glu(D-Trp-OH)—O-Et with potassium carbonate in DMF and R²—I wherein R²—I is methyl iodide gives the compound CBz-D-Glu(D-Trp-O—R²)—O—R¹ wherein R¹ is ethyl and R² is methyl. Hydrogenation of this compound over Pd/C affords the compound H-D-Glu(D-Trp-O-Me)-O-Et.

Compounds of the present invention or salts thereof may be formulated into a pharmaceutical formulation. Many compounds of this invention are generally water soluble and may be formed as salts. In such cases, pharmaceutical compositions in accordance with this invention may comprise a salt of such a compound, preferably a physiologically acceptable salt, which are known in the art. Pharmaceutical preparations will typically comprise one or more carriers acceptable for the mode of administration of the preparation, be it by injection, inhalation, topical administration, lavage, gavage or other modes suitable for the selected treatment. Suitable carriers are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Compounds or pharmaceutical compositions in accordance with this invention or for use in this invention may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition according to the invention includes a therapeutically effective amount or a prophylactically effective amount. A “therapeutically effective amount” refers to an amount effective, at dosages and for periods of time necessary, to achieve the desired therapeutic result, such as improved PASI score or other suitable clinical indication known to a person of skill in the art. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as a desirable PASI score or other suitable clinical indication known to a person of skill in the art. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

As used herein, a “subject” may be a human, non-human primate, rat, mouse, cow, horse, pig, sheep, goat, dog, cat, etc. The subject may be suspected of having or at risk for having psoriasis and/or atopic dermatitis and/or a medical condition wherein an agent is used in modulating the immune system. Diagnostic methods for psoriasis, atopic dermatitis and various disorders for which immune modulating compounds are used and the clinical delineation of those conditions' diagnoses are known to those of ordinary skill in the art.

### EXAMPLES

The following examples are illustrative of some of the embodiments of the invention described herein. These examples do not limit the spirit or scope of the invention in any way.

### Example 1

Preparation of N-alpha-tert-butoxycarbonyl-gamma-D-glutamyl(alpha-benzyl ester)-D-tryptophan methyl ester or (2R)-tert-Butoxycarbonylamino-(4R)-[2-(1H-indol-3-yl)-1-methoxycarbonyl-ethylcarbamoyl]-butyric acid benzyl ester or N-t-Boc-D-Glu-(gamma-D-Trp-OMe)-alpha-OBzl}

### Procedure 1A:

Preparation of a Pure Reference Sample of N-t-Boc-D-Glu-(gamma-D-Trp-OMe)-alpha-OBzl, Using Silica Gel Chromatography Purification.

To a stirred ice-cooled solution of Boc-D-Glu-OBzl (6.00 g, 17.8 mmol) in CH₂Cl₂ (70 mL) was successively added EDC (5.11 g, 26.6 mmol), HOBt (3.60 g, 26.6 mmol) and DIPEA (4.60 mL, 26.6 mmol). Then, a solution of H-D-Trp-OMe.HCl (6.77 g, 26.6 mmol) and DIPEA (4.60 mL, 26.6 mmol) in CH₂Cl₂ (50 mL) was added dropwise. The resulting mixture was stirred at ice-cold temperature (−3° C. to 0° C.) for 1 h, then allowed to warm to room temperature, and stirred for 16 h. The reaction mixture was evaporated to dryness. The residue was partitioned between EtOAc and a saturated solution of NaHCO₃. The organic fraction was collected, washed with 10% citric acid, followed by brine. The organic layer was dried over Na₂SO₄, filtered and concentrated to a thick oil. The residue was purified by column chromatography on silica gel using a solvent gradient of a mixture of hexanes and EtOAc (8/2, 7/3 and 3/7 ratio, v/v) as eluant to afford the title product (9.40 g, 98%) as a white solid. ¹H NMR (DMSO-d₆) δ ppm: 10.86 (s, 1H), 8.31 (d, J=7.4 Hz, 1H), 7.49 (d, J=7.7 Hz, 1H), 7.31-7.35 (m, 7H), 7.14 (d, J=2.0 Hz, 1H), 7.06 (t, J=7.9 Hz, 1H), 6.98 (t, J=6.8 Hz, 1H), 5.12 (q, J=5.9 Hz, 2H), 4.5 (q, J=6.5 Hz, 1H), 3.96-4.03 (m, 1H), 3.55 (s, 3H), 2.98-3.16 (m, 2H), 2.18-2.24 (m, 2H), 1.86-1.95 (m, 1H), 1.71-1.80 (m, 1H), 1.37 (s, 9H); ¹³C NMR (DMSO-d₆) δ ppm: 172.4 (C), 172.3 (C), 171.4 (C), 155.6 (C), 136.1 (C), 136.0 (C), 128.4 (CH), 127.9 (CH), 127.7 (CH), 127.1 (C), 123.6 (CH), 120.9 (CH), 118.4 (CH), 117.9 (CH), 111.4 (CH), 109.5 (C), 78.2 (C), 65.8 (CH₂), 53.3 (CH), 53.16 (CH), 51.7 (CH₃), 31.3 (CH₂), 28.2 (CH₃), 27.1 (CH₂), 26.4 (CH₂); MS (m/z) 538 [M+1]⁺; Anal. Calcd. for C₂₉H₃₅N₃O₇.0.5H₂O: C, 63.72; H, 6.64; N, 7.69. Found: C, 63.79; H, 6.06; N, 7.65.

### Procedure 1B:

Preparation of a Pure Sample of N-t-Boc-D-Glu-(gamma-D-Trp-OMe)-alpha-OBzl by Recrystallization.

A suspension of Boc-D-Glu-OBzl (60.26 g, 178.6 mmol) in CH₂Cl₂ (335 mL) was cooled to ca. −1° C., and stirred for 15 min. Then, DIPEA (46.70 mL, 268.0 mmol), HOBt (36.20 g, 268.0 mmol), EDC (51.38 g, 268.0 mmol) were successively added. Afterwards, a solution of H-D-Trp-OMe.HCl (68.25 g, 268.0 mmol) and DIPEA (46.70 mL, 268.0 mmol) in CH₂Cl₂ (187 mL) was added dropwise. The resulting mixture was stirred at ice-cold temperature (−1° C. to −5° C.) for 2 h, then allowed to warm to room temperature, and stirred for overnight under a blanket of nitrogen.

The reaction mixture was evaporated to dryness. The residue was partitioned between EtOAc (200 mL), a saturated solution of Na₂CO₃ (100 mL) and H₂O (150 mL). The aqueous layer was extracted again with EtOAc (200 mL). The organic fractions were collected, washed with H₂O (100 mL), 10% citric acid (2×200 mL), and brine (60 mL). The organic layer was dried over Na₂SO₄, filtered and evaporated to dryness. The residue was dissolved in EtOAc (141 mL), then hexanes (106 mL) were added. The resultant suspension was stirred for 6 h, and filtered. The solid was thoroughly washed with hexanes (100 mL), then dried under vacuum in an oven at 40° C. for overnight. An off white solid was obtained (81.67 g, 85%). ¹H NMR data conforms to structure (see Example 1, Procedure 1A).

### Procedure 1C:

Preparation of a Pure Sample of N-t-Boc-D-Glu-(gamma-D-Trp-OMe)-alpha-OBzl without Chromatographic Purification and Determination of Synthetic Impurities.

Boc-D-Glu-OBzl (48.0 g, 142.2 mmol) was dissolved in 270 mL of dichloromethane and then cooled to 0-5° C. using an ice bath. HOBt (23.8 g, 156.4 mmol) was added followed by DIPEA (27.0 mL, 156.4 mmol) and stirred for 10 min. EDC (38 g, 199.1 mmol) and a premixed H-D-Trp-OMe solution (prepared from H-D-Trp-OMe.HCl (39.7 g, 156.4 mmol) and DIPEA (27.0 mL, 156.4 mmol) in 150 mL of dichloromethane stirred at room temperature for 20 min) were added successively to the solution. The reaction was stirred at 0° C. for 2 hours and then overnight at room temperature. The reaction was poured over 250 mL of distilled water and extracted. The organic layer was washed with 250 mL each of 10% citric acid, 2× with 5% NaHCO₃ solution and brine. The organic layer was dried over sodium sulfate and concentrated in vacuo to yield a pale yellow foamy solid.

The solid was dissolved in approximately 250 mL of ethyl acetate and evaporated to dryness. The operation was performed twice to form a waxy solid. To the solid material was added 100 mL of ethyl acetate and allowed to stir at room temperature. The mixture was stirred at a moderate to fast speed until a slurry-like suspension had formed—this process takes approximately 45 min (stirring for a prolonged period of time can result in solidification of the solution into a gelatin-like substance). Then, 75 mL of hexanes was added and the mixture was stirred for an additional 10 min. At this point, another 20 mL of ethyl acetate was added and the slurry was filtered immediately to give a fluffy pale pink solid. The solid was washed immediately three times with 30 mL of hexanes, which helped to remove the pinkish color. The filtrate was collected and was allowed to sit undisturbed for 40 minutes. A granular solid had precipitated out from the filtrate. The mixture was filtered and the solid was washed three times with 10 mL of hexanes.

The filtrate was collected and concentrated to a solid. The solid was dissolved in 20 mL of ethyl acetate and stirred until a slurry had formed. Then, 40 mL of hexanes was added and the mixture stirred for 5 min. The mixture was filtered and the collected solid was washed with hexanes. The combined solids were dried overnight in an oven (35° C.) under vacuum to constant weight. Thus, 59.0 g (77.2%) of the title compound was obtained. Mp: 83.1-87.5° C.; ¹H NMR data were identical to those described in Example 1, Procedure 1A; HPLC purity (PEAK AREA PERCENT): 97.2%; Retention time: 7.56 min; HPLC Conditions: Column Waters Symmetry C 18, 3.9×150 mm, 5 μm; Mobile phase: 0.035% HClO₄, pH 2/CH₃CN, gradient (min-% CH₃CN) 0-35, 10-90, 12-90; Flow rate: 1 mL/min; λ: 230, 260, 280 nm

Analysis of impurities in the mother liquor.

The result of the analysis of the mother liquor by TLC (50/50 EtOAc/Hexanes) is shown in the table below. Spots A and B are both UV active, but gave negative ninhydrin tests. The product and baseline spots both gave positive ninhydrin tests. Samples of A and B have been isolated by column chromatography on silica gel, and their structures have been elucidated by ¹H NMR and MS/MS. The structures of A and B are shown below. Rf Values in 50/50 EtOAc/Hexanes:

| Spot | Rf value |
|---|---|
| A | 0.80 |
| B | 0.60 |
| Product | 0.39 |

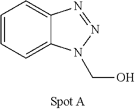

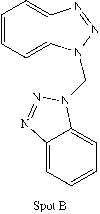

Both spots A and B are impurities associated with HOBt. The impurities can be removed by recrystallization. Any trace impurities can be removed in the subsequent hydrolysis step.

### Example 2

### Preparation of N-alpha-tert-butoxylcarbonyl-D-isoglutamyl-D-tryptophan or (2R)-tert-butoxycarbonylamino-(4R)-[1-carboxy-2-(1H-indol-3-yl)-ethylcarbamoyl]-butyric acid or N-t-alpha-Boc-D-iGlu-D-Trp-OH

### Procedure 2A:

One Phase Hydrolysis with NaOH.

To a stirred solution of N-t-Boc-D-Glu-(gamma-D-Trp-OMe)-alpha-OBzl (3.7 g, 6.9 mmol) from Example 1, Procedure 1A in MeOH (40 mL), was added a solution of NaOH (1.0 g, 25 mmol) in H₂O (20 mL). The resulting solution was stirred at room temperature overnight. The reaction mixture was poured into a 1N solution of NaOH (100 mL), and the aqueous mixture was washed with EtOAc (2×100 mL). The aqueous layer was acidified with a 3N HCl solution, then extracted with EtOAc (2×50 mL). The organic fractions were combined, dried over Na₂SO₄ and evaporated to dryness under reduced pressure. A white solid was obtained (2.7 g, 91%). M.p. 148-158° C.; ¹H NMR (DMSO-d₆) δ ppm: 12.47 (br, 2H), 10.82 (s, 1H), 8.21 (d, J=7.8 Hz, 1H), 7.53 (d, J=7.8 Hz, 1H), 7.34 (d, J=8.1 Hz, 1H), 7.13 (d, J=2.0 Hz, 1H), 7.06 (t, J=7.5 Hz, 2H), 6.98 (t, J=7.4 Hz, 1H), 4.46 (q, J=5.3 Hz, 1H), 3.88-3.83 (m, 1H), 3.17-2.97 (dd, J=5.2 and 8.4 Hz, 2H), 2.23-2.10 (m, 2H), 1.90-1.82 (m, 1H), 1.75-1.68 (m, 1H), 1.38 (s, 9H); ¹³C NMR (DMSO-d₆) δ ppm: 173.9 (C), 173.4 (C), 171.5 (C), 155.6 (C), 136.1 (C), 127.2 (C), 123.5 (CH), 120.9 (CH), 118.4 (CH), 118.2 (CH), 111.4 (CH), 109.9 (C), 78.0 (C), 53.1 (CH), 52.9 (CH), 31.7 (CH₂), 28.2 (CH₃), 27.2 (CH₂), 26.7 (CH₂); FT-IR (KBr) ν: 3415, 3338, 2986, 1719, 1686, 1654, 1534, 1424, 1366, 1252, 1169, 1069, 744, 634, 429 cm⁻¹; MS (m/z) 434 [M+1]⁺.

### Procedure 2B:

One Phase Hydrolysis with LiOH.

To a stirred ice-cooled (0° C. to 5° C.) solution of Boc-D-Glu-(gamma-D-Trp-OCH₃)-alpha-OBzl (46.06 g, 85.68 mmol) in MeOH (200 mL) was added a solution of LiOH (10.78 g, 257.0 mmol) in H₂O (136 mL). The resulting solution was stirred and maintained between 0° C. to 10° C. for 3 h. The reaction mixture was poured into a saturated solution of Na₂CO₃ (100 mL) and H₂O (150 mL), the aqueous mixture was washed with EtOAc (2×150 mL). The aqueous layer was acidified to pH=2-3 with a 3N HCl solution, then extracted with EtOAc (2×200 mL). The organic fractions were combined, dried over Na₂SO₄ and evaporated to dryness under reduced pressure. A white solid was obtained (36.65 g, 98.7%). ¹H NMR and MS/MS data conform to structure (see Example 2, Procedure 2A).

### Procedure 2C:

Preparation of Boc-D-iGlu-D-Trp-OH Without Chromatographic Purification Using a Two Phase Hydrolysis Process.

Lithium hydroxide (4.1 g, 97.7 mmol) was dissolved in 35 mL of distilled water. Then, 65 mL of methyl t-butyl ether (MTBE) was added followed by the dipeptide, Boc-D-Glu-(gamma-D-Trp-OCH₃)-alpha-OBzl, (25 g, 46.5 mmol) obtained as described in Example 1. A very thick suspension formed immediately and 15 mL of methanol and 15 of MTBE were added under vigorous stirring. An additional 2 mL of methanol was added and the solids slowly dissolved over approximately 5 min. Once all material had dissolved, the solution was a yellow/green color with the top organic phase having a pale green color and the aqueous phase a yellow color. The reaction was allowed to stir vigorously at room temperature for 80 min, at which time, no starting material remained in the organic phase and the aqueous phase contained the product (TLC monitoring: 1/1 EtOAC/Hexanes, v/v). The solution was poured into a separatory funnel and the 2 phases separated. The organic phase was washed with 15 mL of water. The organic phase turned pink upon washing with water. The combined aqueous phase was washed twice with 30 mL of ethyl acetate. The aqueous phase was acidified to ca. pH 2 by adding dropwise 16.6 mL of 6N hydrochloric acid at room temperature. The aqueous phase was extracted twice with 50 mL of ethyl acetate. A minimum amount of methanol was added during the second extraction to help with solubility of the product in the organic layer. The combined organics were dried over sodium sulfate and concentrated in vacuo from a yellow liquid to yield a white solid. The solid was dried overnight in an oven (28° C.) under vacuum to constant weight.

Thus, 18.6 g (92% yield) of the title compound was obtained. Mp: 179.0-184.6° C.; ¹H NMR data were identical to those described in Example 2A; HPLC purity (peak area percent): 98.3%; Retention time: 5.33 min; HPLC Conditions: Column Waters Symmetry C 18, 3.9×150 mm, 5 μm; Mobile phase: 0.035% HClO₄, pH 2/CH₃CN, gradient (T in min-% CH₃CN) 0-20, 10-90, 12-90; Flow rate: 1 mL/min; λ: 230, 260, 280 nm.

The above reaction can be monitored by HPLC. In Procedure 2C above, the presence of benzyl alcohol, arising from the hydrolysis of the benzyl ester moiety, can be observed on TLC (1/1 EtOAC/Hexanes, v/v, as eluant). Benzyl alcohol derives from the hydrolysis of the benzyl ester moiety. The first spot impurity is the same as in Example 1, Procedure 1C. Monitoring by HPLC and analysis by LC/MS indicated that Boc-D-Glu-(gamma-D-Trp-OCH₃)-alpha-OBzl first reacted with the base and methanol to give Boc-D-Glu-(gamma-D-Trp-OCH₃)-alpha-OCH₃, which then quickly hydrolyzed to give the diacid, Boc-D-Glu-(gamma-D-Trp-OH)—OH or Boc-D-iGlu-D-Trp-OH.

### Example 3

### Preparation of D-isoglutamyl-D-tryptophan

### Procedure 3A:

Preparation of D-isoglutamyl-D-tryptophan and its Purification by Recrystallization.

Boc-D-iGlu-D-Trp-OH (20.0 g, 46.14 mmol, from Example 2) was placed in a 1 L-3-necked round bottom flask equipped with a mechanical stirrer. Ethyl acetate (300 mL) was added, and the resulting suspension was cooled to −10° C. in an ice-salt bath. HCl gas was bubbled into the cold suspension. A temperature range of −4° C. to −10° C. was maintained during the course of the reaction, and the progress of the reaction was monitored by HPLC. At a certain point the heterogeneous reaction mixture changed to a clear light pink homogenous solution. And after the starting material was consumed, the reaction mixture became to a suspension again. Volatile materials were then removed in vacuo to give a light pink solid. The solid was dissolved in 60 mL of deionized water, and the resulting solution was washed with dichloromethane (2×25 mL). The pH of the aqueous solution was then brought to ca. 3.0 by adding NaOH (10M, ca. 3.6 mL) under cooling. The resulting solution was filtered to remove any residual solid particulates. The filtrate was collected and stirred vigorously as a solid separated. The solid was collected by filtration. The filtrate was set aside for later use. The solid was then placed back in a round bottom flask, and 30 mL of deionized water was added. The mixture was stirred vigorously, and the solid was collected by filtration. The filtrate was set aside for later use. The solid was then washed with ice-cold deionized water (4×15 mL). The third aqueous wash solution was chloride free, as confirmed by a negative AgNO₃ test (a 4% solution was used). The solid was air dried, then placed in a vacuum oven at 36° C. overnight to give 8.5 g (HPLC purity (peak area percent): 98.3%).

Filtrates from the above steps were combined, and the same recrystallization procedure was carried out to give an additional 3.2 g of the product (HPLC purity (peak area percent): 98.7%). The combined yield of the 2 crops is 11.7 g (75%).

Further treatment of the final filtrate afforded a third crop (1.0 g, HPLC purity (peak area percent): 82.0%).

¹H NMR (D₂O—NaOD, pH 7.0) δ ppm: 7.64 (d, J=7.9 Hz, 1H), 7.43 (d, J=8.1 Hz, 1H), 7.19-7.16 (m, 2H), 7.10 (t, J=7.4 Hz, 1H), 4.52-4.48 (m, 1H), 3.48 (t, J=6.1 Hz, 1H), 3.34-3.29 (m, 1H), 3.08-3.02 (m, 1H), 2.30-2.17 (m, 2H), 1.92-1.75 (m, 2H). HPLC method: Column: XTerra MS C18; 5 μm, 4.6×250 mm; Mobile phase: A=the aqueous phase: 4 mM Tris, 2 mM EDTA, pH 7.4; B=the organic phase: CH₃CN; the gradient program: B %: 0 min. 5%, 15 min. 55%, 30 min. 55%, 32 min. 5%, 40 min. 5%. Flow rate=1 mL/min; Injection volume=5 μL; λ: 222, 254, 282, 450 nm; Retention time of the product=6.4 min.

### Procedure 3B:

Ethyl acetate (250 mL), pre-cooled to 0° C., was saturated with HCl gas for 25 min. Boc-D-iGlu-D-Trp-OH (15.0 g, 34.6 mmol) was added, and a suspension formed. The solution was stirred for 90 min at ice bath temperature. The solvent was evaporated in vacuo to form a white solid. The solid was dissolved in 35 mL of distilled water, forming a thick light brown solution. The aqueous layer was washed twice with 30 mL of dichloromethane, then transferred to a 100 mL beaker. Using a pH electrode to monitor acidity, the pH was adjusted from 1.28 to 2.96 using 3.2 mL of 10N NaOH. The solution was stirred for 1 h at room temperature and a white precipitate slowly formed. The solid was collected by suction filtration, and thoroughly washed with water. The crude solid was suspended in 20 mL of distilled water and left stirring for 2 h at room temperature. The mixture was filtered, the solid was collected and dried to constant weight in an oven under vacuum overnight (40° C.). Thus, 8.6 g (74.5% yield) of the title compound was obtained. HPLC purity (peak area percent): 98.8% Retention time: 4.21 min; HPLC Conditions: Column Waters Symmetry C 18, 3.9×150 mm, 5 μm; Mobile phase: 0.035% HClO₄, pH 2/CH₃CN, gradient (T in min-% CH₃CN) 0-10, 10-90, 12-90; Flow rate: 1 mL/min; λ: 230, 260, 280 nm; ¹H NMR data conforms to structure.

### Example 4

### Synthesis of D-isoglutamyl-D-tryptophan mono ammonium salt (1:1) and its Isolation by Column Chromatography Purification

### Procedure 4A:

HCl gas was bubbled into a stirred ice-cooled (0° C. to 5° C.) solution of Boc-D-Glu-(-D-Trp-OH (2.5 g, 5.8 mmol) in EtOAc (60 mL) for 2.5 h. The reaction mixture was then evaporated to dryness. Purification of the residue by column chromatography on silica gel using a solvent gradient of a mixture of isopropanol and ammonium hydroxide (28-30% NH₄OH) (8/2 and 7/3 ratio, v/v) as eluant afforded the title product (1.8 g, 84.7%) as a white solid after solvent evaporation. M.p. 124-128° C.; ¹H NMR (DMSO-d₆) δ ppm: 10.98 (s, 1H), 8.25 (d, J=5.9 Hz, 1H), 7.53 (7.8 Hz, 1H), 7.30 (d, J=8.0 Hz, 1H), 7.17 (s, 1H), 7.00 (t, J=7.7 Hz, 1H), 6.92 (t, J=7.2 Hz, 1H), 4.28 (m, 1H), 3.22-3.31 (m, 2H), 2.90-2.96 (m, 1H), 2.23-2.25 (m, 2H), 1.97-1.98 (m, 1H), 1.84-1.86 (m, 1H); ¹³C NMR (DMSO-d₆) δ ppm: 175.5 (C), 171.6 (C), 171.4 (C), 136.0 (C), 127.6 (C), 123.5 (CH), 120.5 (CH), 118.3 (CH), 117.9 (CH), 111.5 (C), 111.3 (CH), 55.3 (CH), 53.7 (CH), 32.5 (CH₂), 27.8 (CH₂), 27.4 (CH₂); ¹⁴N NMR (D₂O) δ ppm: 20.4 (s); FT-IR (KBr) ν: 3406, 3055, 1581, 1456, 1399, 1341, 1096, 1009, 744, 535, 426 cm⁻¹; MS (m/z) 334 [Diacid+1]⁺; Anal. Calcd. for C₁₆H₂₂N₄O₆.H₂O: C, 52.17; H, 6.57; N, 15.21. Found: C, 51.95; H, 6.84; N, 14.85. The substance is the mono ammonium salt of D-isoglutamyl-D-tryptophan (1:1). This material is amorphous as confirmed by XRPD.

### Procedure 4B:

HCl gas is condensed into cold ethyl acetate (133.4 g) at −2° C. (external ice bath temperature) for 16 minutes. The weight increase of the solution is 21 g. Boc-D-iGlu-D-Trp-OH (3.8 g, 8.73 mmol) was dissolved in 50 mL of the above solution. The resulting mixture was maintained between 0 and 5° C. for 55 minutes. The reaction was monitored by TLC, then evaporated under reduced pressure (rotary evaporator temperature: 51-52° C.) to dryness. Purification by flash chromatography on silica gel using a solvent gradient of a mixture of isopropanol and ammonium hydroxide (28-30%) (8/2 and 7/3 ratio, v/v) as eluant afforded the product (2.0 g, 62%) as an off-white solid. The ¹H NMR data are similar to those reported in Example 4, Procedure 4A. This material is amorphous as confirmed by XRPD.

### Example 5

### Synthesis of D-isoglutamyl-D-tryptophan, mono ammonium salt (1:1)

### Procedure 5A:

Synthesis of D-isoglutamyl-D-tryptophan, mono ammonium salt (1:1) by Removal of Inorganic Salts Using AMBERLYST®15 Resin Followed by Column Chromatographic Purification.

HCl gas was bubbled into a stirred ice-cooled (0° C. to 5° C.) suspension of Boc-D-iGlu-D-Trp-OH obtained (10.82 g, 24.96 mmol) in EtOAc (200 mL) for 2 h. The reaction mixture was then evaporated to dryness. The residue was dissolved in H₂O (30 mL), and neutralized to pH=6-7 with 6N NaOH. The resulting solution was loaded onto a chromatography column packed with the AMBERLYST®15 resin, followed by elution of H₂O until pH=5-5.5, then 100% isopropanol (pH=7) and finally 25% NH₄OH/IPA (pH=10).

Fractions containing the product were combined and evaporated to dryness under reduced pressure. Further purification of the residue by column chromatography on silica gel using a solvent gradient of a mixture of isopropanol and conc. ammonium hydroxide (17/3, 4/1 and 7/5 ratio, v/v) as eluant afforded the title product (6.68 g, 72.7%) as a light yellow foamy solid. ¹H NMR and MS/MS data conform to structure (see Procedure 4A). The water content as determined by the Karl-Fisher test was 3.7%.

### Procedure 5B:

Synthesis of D-isoglutamyl-D-tryptophan, mono ammonium salt (1:1) by Removal of Inorganic Salts Using Amberlyst15 Resin Followed by purification by recrystallization.

To a stirred ice-cooled (0° C. to 5° C.) suspension of Boc-D-iGlu-D-Trp-OH (10.75 g, 24.80 mmol) in EtOAc (200 mL) was bubbled HCl gas. The reaction mixture was maintained in the ice bath (0° C. to 5° C.) for 2 h. TLC analysis (30% ammonia in isopropanol) showed the complete conversion of the starting material. The reaction mixture was evaporated to dryness in vacuo, the residue was dissolved in H₂O (30 mL), and neutralized to pH=6-7 with 10N NaOH. The resulting homogenous solution was loaded onto a chromatography column packed with AMBERLYST®15 resin, followed by elution of H₂O (2450 mL) until pH=4-5.5, isopropanol (1000 mL) and 25% NH₄OH/isopropanol. The fractions containing the product were combined and concentrated to dryness. A colorless foamy solid was obtained, to which was added isopropanol (150 mL) and H₂O (30 mL). The resulting suspension was stirred at room temperature overnight. The solid was collected by suction filtration, thoroughly washed with isopropanol (2×60 mL), then EtOAc (2×60 mL), and finally dried under vacuum in an oven at 42° C. for overnight. An off white solid was obtained (6.60 g, 72.2%). ¹H NMR and MS/MS data conform to structure (see example 5). The water content as determined by the Karl-Fisher test was 5.9%.

### Example 6

### Synthesis of D-isoglutamyl-D-tryptophan, mono ammonium salt (1:1) from H-D-iGlu-D-Trp-OH

### Procedure 6A:

Preparation of D-isoglutamyl-D-tryptophan, Mono Ammonium salt (1:1) Using CBz-D-Glu-(gamma-D-Trp-OH)-gamma-OBzl as an Intermediate.

EDC (562 mg, 2.93 mmol) was added to a solution of Z-D-Glu-OBz (990 mg, 2.67 mmol) and N-hydroxysuccinimide (337 mg, 2.93 mmol) in DMF (50 mL) at ice-water bath, and the resulting clear solution was stirred for overnight at RT. H-D-Trp-OH (640 mg, 3.13 mmol) and Et₃N (1 mL) was added at RT. After 20 minutes, the material was mixed with water and extracted with ethyl acetate. The combined EtOAc was washed with 10% citric acid and followed by brine, dried over Na₂SO₄, filtered, evaporated to dryness, dried under vacuum to give 1.49 g of the CBz-D-iGlu-(gamma-OBzl)-D-Trp-OH. This material was hydrogenated with 33% (w/w) of 10% Pd/C at atmospheric pressure. After four hours, the catalyst was filtered through CELITE® and the filtrate was evaporated to give an oil. The crude product was purified by flash column chromatography using isopropanol/NH₄OH (80/20 to 70/30, v/v) as eluant to give the title compound (813 mg). MS/MS and ¹H NMR data are similar to the compound obtained using the method shown in Example 4 above.

### Procedure 6B:

Preparation of D-isoglutamyl-D-tryptophan, Mono Ammonium Salt (1:1) from H-D-iGlu-D-Trp-OH (see Example 3).

H-D-iGlu-D-Trp-OH (1 g from Example 3) was mixed with ammonium hydroxide (0.55M, 6 mL). The mixture was stirred and the pH was measured to be around 4.5. Ammonium hydroxide (0.55M) was added dropwise until the pH of the solution reached between 7.0 to 7.5. Volatile materials were removed in vacuo, and the residual oil was mixed with isopropanol. A white precipitate appeared. After 2 h, the solid ammonium salt was collected by suction filtration. The solid was dried to constant weight (1 g) under high vacuum for 12 h to give the D-isoglutamyl-D-tryptophan, ammonium salt (1:1). The water content as determined by the Karl-Fisher test was 4.6%.

### Example 7

### Purification of H-D-iGlu-D-Trp-OH by Column Chromatography on Silica Gel Using a Mixture of Isopropanol and Water

### A. Preparation of Cbz-D-Glu-(gamma-D-Trp-OBzl)-α-OBzl

To an ice-cooled solution of 2.67 g of Cbz-D-Glu-OBzl in 50 mL of DMF was added 0.91 g of N-hydroxysuccinimide (1.1 equiv.) and 1.52 g of EDC (1.1 equiv.), and the resulting solution was stirred at ice-water bath for 1 h and then at RT for overnight. To this reaction mixture was added 2.50 g of H-D-Trp-OBzl.HCl (1.05 equiv.) and 3 mL of Et₃N at RT. The reaction was complete after 1 h as monitored by HPLC.

The reaction mixture was quenched with deionized-water at ice-water bath, and then extracted with EtOAc several times. The combined EtOAc extracts was washed with 10% citric acid, followed by brine, dried over Na₂SO₄, filtered, and evaporated to dryness. The residue was coated onto silica gel in MeOH and the mixture was concentrated in vacuo. The latter was applied on top of a wet-packed silica gel column and the desired product, Cbz-D-Glu-(gamma-D-Trp-OBzl)-α-OBzl, was eluted using a solvent gradient mixture (EtOAc/Hexanes, from 80/20 to 100/0). The desired fractions were combined and concentrated in vacuo to afford 4.64 g (99.6% yield) of title compound. HPLC purity (peak area percent): 93%; HPLC conditions: Column: Symmetry C18, 3.9×150 mm, 5 μm; Mobile phase: 0.035% HClO₄ (pH=2.5)/CH₃CN=gradient (min-CH₃CN %: 0-10, 10-100, 12-100, 14-50); Flow rate: 1 mL/min; λ: 280 nm; Retention time: 9.7 min.

P B. Purification of D-isoglutamyl-D-tryptophan by Column Chromatography using a Mixture of Isopropanol and Water.

To a suspension of 4.0 g of Cbz-D-Glu-(gamma-D-Trp-OBzl)-α-OBzl prepared as described in Example 7A above in 150 mL of 95/5 MeOH/H₂O (v/v) was added 1.5 g of Pd/C (37.5% w/w). The mixture was hydrogenated under 30 psi hydrogen pressure. The reaction was done after 75 min as monitored by HPLC. The catalyst was filtered off over a bed of CELITE® and the filtrate was concentrated under reduced pressure at 45° C. The residue was then purified by flash chromatography on silica gel using a mixture of isopropanol/H₂O (80/20 ratio, v/v). The most pure fractions (by HPLC) were combined together and concentrated in vacuo. Thus, the title compound (1.1 g, 52%) was obtained as a light yellow powder. HPLC purity (peak area percent): 99%. MS/MS and ¹H NMR conformed to the desired structure.

Less pure fractions (by HPLC) were combined together and concentrated in vacuo, and ca. another 1.0 g (48% yield) of the title compound was obtained. HPLC purity (peak area percent): 96.7%. MS/MS and ¹H NMR conformed to the desired structure.

HPLC conditions are the same as in section 7A above; Retention time of H-D-iGlu-D-Trp-OH is 4.0 min.

### Example 8

### Preparation of 2-(3-amino-2,6-dioxo-piperidin-1-yl)-3-(1H-indol-3-yl)-propionic acid

The titled compound is a possible degradation product of H-D-iGlu-D-Trp-OH. It is independently synthesized and used as a reference in the HPLC analysis of products described in Examples 3 to 7 above.

To an ice-cooled solution of Z-D-Glu-OEt (1 g, 3.23 mmol) in DMF (60 mL), was added N-hydroxysuccinimide (409 mg, 3.56 mmol), EDC (682 mg, 3.56 mmol), and the resulting solution was stirred at ice-water bath for 1 h and then RT for overnight. To this reaction mixture was added H-D-Trp-OH (792 mg, 3.88 mmol) and Et₃N (1 mL) at RT. Water was added after 1.5 h at ice bath temperature. The mixture was extracted with EtOAc several times. The combined EtOAc extracts was washed with 10% citric acid, followed by brine, dried over Na₂SO₄, filtered, concentrated to dryness, and dried under vacuum to afford 410 mg of crude product fraction A. The aqueous layer was concentrated in vacuo at a bath temperature of 55° C. The residue was dissolved in CH₂Cl₂, and the organic fraction was washed with 10% citric acid (1×20 mL) and brine, dried over Na₂SO₄, filtered, concentrated to almost dryness at 50° C. to give crude product fraction B. Both crude product fractions A and B were combined and hydrogenated over 10% Pd/C (40% w/w of Pd/C) under atmospheric pressure using a hydrogen-filled balloon at room temperature for 2.75 hours. The reaction mixture was filtered through CELITE®. The filtrate contained the product H-D-iGlu(gamma-OEt)-D-Trp-OH, and was analyzed by HPLC (same method as the one described in 7A above). The product peak has a retention time of 4.54 min. The filtrate was concentrated to dryness under reduced pressure at a bath temperature of ca. 45° C. HPLC analysis showed the partial conversion of the product with Rₜ at 4.54 min to another peak with Rₜ at 4.45 min. The crude product was purified by flash column chromatography using isopropanol and conc. NH₄OH (elution gradient: 90/10 to 80/20, v/v). HPLC analysis of this material (670 mg) showed a peak with Rₜ at 4.45 min. Structure elucidation by 1H NMR spectroscopy indicated that the product after workup was the cyclized Compound shown below:

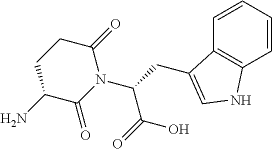

¹H NMR (CD₃OD) δ ppm: 7.60 (d, J=7.8 Hz, 1H), 7.31 (d, J=8.0 Hz, 1H), 7.10 (s, 1H), 7.07 (t, J=7.8 Hz, 1H), 7.00 (t, J=7.4 Hz, 1H), 4.68-4.65 (m, 1H), 4.05-4.02 (m, 1H), 3.44 (dd, J=14.6 Hz, J=4.6 Hz, 1H), 3.23-3.18 (m, 1H), 2.34-2.24 (m, 1H), 2.13-2.05 (m, 1H), 2.00-1.92 (m, 1H) and 1.76-1.68 (m, 1H); MS (m/z): 316 [M+1]⁺, 188 (100%).

HPLC Method: Column—Symmetry C18, 5 μm, 3.9×150 mm, WAT 046980; Mobile phase—0.035% HClO₄/CH₃CN, gradient; Method: min—CH₃CN %: 0-10%, 10-100%, 12-100%, 14-50%; Flow rate: 1.0 mL/min; Detection λ: 254 nm.

### Example 9

### Synthesis of L-isoglutamyl-L-tryptophan, Mono Ammonium Salt (1:1)

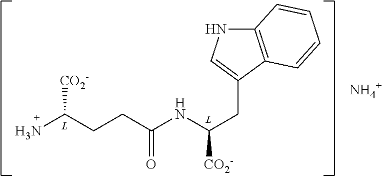

### A: Synthesis of Boc-L-Glu-(gamma-L-Trp-O-t-Bu)-alpha-O-t-Bu

A solution of Boc-L-Glu-O-t-Bu (1.50 g, 4.9 mmol) in CH₂Cl₂ (50 mL) was cooled to −3° C., and stirred for 15 min. Then, 1-hydroxybenzotriazole (HOBt, 1.00 g, 7.4 mmol), N-(3-dimethylaminopropyl)-N′-ethylcarbodiimide hydrochloride (EDC, 1.42 g, 7.4 mmol) and diisopropylethylamine (DIPEA, 1.30 mL, 7.4 mmol) were successively added. Afterwards, a solution of H-L-Trp-O-t-Bu.HCl (2.20 g, 7.4 mmol) and DIPEA (1.30 mL, 7.4 mmol) in CH₂Cl₂ (20 mL) was added dropwise. The resulting mixture was stirred at ice-cold temperature (−3° C. to 0° C.) for 1 h, then allowed to warm to room temperature, and stirred for overnight under a blanket of nitrogen.

The reaction mixture was evaporated to dryness. The residue was partitioned between EtOAc (40 mL) and a saturated solution of NaHCO₃ (100 mL). The organic fraction was collected, washed with 10% citric acid, followed by brine (30 mL). The organic layer was dried over Na₂SO₄, filtered and concentrated to a thick oil. Purification of the residue by column chromatography on silica gel using a solvent gradient of a mixture of hexanes and EtOAc (85/15, 80/20 and 60/40 ratio, v/v) as eluant afforded the title product (2.55 g, 95%) as a white solid. ¹H NMR (DMSO-d₆) δ ppm: 10.84 (s, 1H), 8.18 (d, J=7.5 Hz, 1H), 7.52 (d, J=7.7 Hz, 1H), 7.35 (d, J=8.0 Hz, 1H), 7.12-7.15 (m, 2H), 7.06 (t, J=7.6 Hz, 1H), 6.98 (t, J=6.8 Hz, 1H), 4.41 (q, J=6.7 Hz, 1H), 3.73-3.80 (m, 1H), 2.94-3.12 (m, 2H), 2.13-2.21 (m, 2H), 1.60-1.85 (m, 2H), 1.28-1.38 (m, 27H); ¹³C NMR (DMSO-d₆) δ ppm: 171.5 (C), 171.4 (C), 171.1 (C), 155.5 (C), 136.1 (C), 127.2 (C), 123.5 (CH), 120.9 (CH), 118.3 (CH), 118.1 (CH), 111.3 (CH), 109.7, 80.2, 78.0, 53.9 (CH), 53.6 (CH), 31.5 (CH₂), 28.2 (CH₃), 27.9 (CH₃), 27.6 (CH₃), 27.5 (CH₃), 27.2 (CH₂), 26.6 (CH₂); MS (m/z) 546 [M+1]⁺; Anal. Calcd. for C₂₉H₄₃N₃O₇.0.5H₂O: C, 62.80; H, 8.00; N, 7.58. Found: C, 62.69; H, 8.56; N, 7.57.

### B: Synthesis of L-isoglutamyl-L-tryptophan, Mono Ammonium Salt (1:1)

HCl gas was bubbled into a stirred ice-cooled (0° C. to 5° C.) solution of Boc-L-Glu-((-L-Trp-O-t-Bu)-gamma-O-t-Bu obtained as described above (2.38 g, 4.4 mmol) in CH₂Cl₂ (40 mL) for 5 h. The reaction mixture became cloudy during HCl gas bubbling. The temperature of the reaction was kept at below 30° C. with ice cooling. The reaction mixture was then evaporated to afford a white solid (crude material weight=2.80 g).

Purification of a crude sample (482 mg) using reversed phase high performance flash chromatography (HPFC™) (Biotage) with a C18HS M+40 column and a solvent gradient of a mixture of 15 mM NH₄OAc and CH₃CN as eluant afforded the title compound after solvent evaporation and freeze-drying the material (150 mg). ¹H NMR (DMSO-d₆) δ ppm: 7.57 (d, J=7.8 Hz, 1H), 7.39 (d, J=8.1 Hz, 1H), 7.11-7.14 (m, 2H), 7.05 (t, J=7.2 Hz, 1H), 4.44-4.48 (m, 1H), 3.42 (t, J=5.7 Hz, 1H), 3.25 (dd, J=14.7, 4.7 Hz, 1H), 2.97-3.03 (m, 1H), 2.14-2.19 (m, 2H), 1.72-1.85 (m, 2H); FT-IR (KBr) ν: 3057, 1581, 1400, 745 cm⁻¹; MS (m/z) 334 [diacid+1]⁺; Anal. Calcd. for C₁₆H₂₂N₄O₆.H₂O: C, 52.17; H, 6.57; N, 15.21. Found: C, 51.92; H, 6.80; N, 14.94. The substance is the mono ammonium salt of L-isoglutamyl-L-tryptophan (1:1).

### Example 10

### Synthesis of H-D-isoglutamyl-L-tryptophan, Mono Ammonium Salt (1:1)

### A. Synthesis of Boc-D-Glu-(gamma-L-Trp-O-t-Bu)-alpha-O-t-Bu

The procedure described in Example 1A was used. To a stirred ice-cooled solution of Boc-D-Glu-O-t-Bu (4.00 g, 13.2 mmol) in CH₂Cl₂ (75 mL) were successively added EDC (3.80 g, 19.8 mmol), HOBt (2.68 g, 19.8 mmol) and DIPEA (3.50 mL, 19.8 mmol). The resulting mixture was stirred at ice-cold temperature for 20 min. Then, a solution of H-L-Trp-O-t-Bu.HCl (5.88 g, 19.8 mmol) and DIPEA (3.50 mL, 19.8 mmol) in CH₂Cl₂ (50 mL) was added dropwise over a period of 10 min. The resulting mixture was stirred at ice-cold temperature for 1 h, then allowed to warm to room temperature, and stirred for overnight.

The reaction mixture was evaporated to dryness. The residual thick oil was taken up in EtOAc (50 mL), and the organic layer was successively washed with a saturated NaHCO₃ solution (100 mL), a 10% citric acid solution (100 mL), brine (100 mL), and water (100 mL). The organic layer was dried over Na₂SO₄, filtered and concentrated in vacuo. Purification of the residue by column chromatography on silica gel using a solvent gradient of a mixture of hexanes and EtOAc (8/2, 7/3 and 6/4 ratio, v/v) as eluant afforded the title product (5.88 g, 82%) as an off white solid. ¹H NMR (DMSO-d₆) δ ppm: 10.85 (s, 1H), 8.18 (d, J=7.4 Hz, 1H), 7.50 (d, J=7.7 Hz, 1H), 7.33 (d, J=8.0 Hz, 1H), 7.14-7.05 (m, 3H), 6.99 (t, J=7.1 Hz, 1H), 4.40 (q, J=7.5 Hz, 1H), 3.81-3.75 (m, 1H), 3.12-3.07 (m, 1H), 3.01-2.95 (m, 1H), 2.18-2.15 (m, 2H), 1.86-1.83 (m, 1H), 1.73-1.65 (m, 1H), 1.39-1.29 (m, 27H); MS (m/z) 568 [M+Na]⁺; 546 [M+1]⁺; Anal. Calcd. for C₂₉H₄₃N₃O₇.0.75H₂O: C, 62.29; H, 8.02; N, 7.51. Found: C, 62.43; H, 7.95; N, 7.08.

### B. Synthesis of H-D-Glu-(-L-Trp-OH, Mono Ammonium Salt (1:1)

The procedure described in Example 1B was used. HCl gas was bubbled into a stirred ice-cooled (about −5° C.) solution of Boc-D-Glu-(gamma-L-Trp-O-t-Bu)-alpha-O-t-Bu obtained as described above (1.59 g, 2.91 mmol) in EtOAc (100 mL) for 45 min. The solution turned from colorless to cloudy yellow. The mixture was stirred at ice-cold temperature for 1 h, then allowed to warm to room temperature, and stirred for another 2 h. The reaction was completed as monitored by HPLC (column: Waters C18, 3.9×150 mm, WAT046980, mobile phase: solvent gradient of a mixture of 0.035% HClO₄ (pH=2-2.5) and acetonitrile, flow rate: 1 mL/min, 8: 210-270 nm).

The reaction mixture was concentrated under reduced pressure to a solid. The solid was dissolved in acetone, and the volatiles were removed under reduced pressure. The latter procedure was repeated two more times. Purification of the residue by column chromatography using a solvent gradient of a mixture of isopropanol and ammonium hydroxide (28-30% NH₄OH) (85/15 and 70/30 ratio, v/v) as eluant afforded the title product (0.42 g, 39%) as an off-white foam. Mp 120-130° C.; ¹H NMR (D₂O) δ ppm: 7.67 (d, J=7.9 Hz, 1H), 7.46 (d, J=8.2 Hz, 1H), 7.18-7.22 (m, 2H), 7.13 (t, J=7.1 Hz, 1H), 4.53 (q, J=3.8 Hz, 1H), 3.45 (t, J=5.8 Hz, 1H), 3.33 (dd, J=14.7 and 4.75 Hz, 1H), 3.07 (dd, J=14.7 and 8.8 Hz, 1H), 2.19-2.31 (m, 2H), 1.78-1.98 (m, 2H); ¹³C NMR (D₂O) δ ppm: 181.4 (C), 176.6 (C), 176.5 (C), 138.8 (C), 129.9 (C), 126.9 (CH), 124.5 (CH), 121.9 (CH), 121.4 (CH), 114.5 (CH), 113.2 (C), 58.6 (CH), 56.7 (CH), 34.2 (CH₂), 30.3 (CH₂), 28.9 (CH₂); MS (m/z) 334 [Diacid+1]⁺.

### Example 11

### Synthesis of L-isoglutamyl-D-tryptophan, Mono Ammonium Salt (1:1)

### A. Synthesis of Boc-L-Glu-((-D-Trp-OMe)-alpha-O-t-Bu

The procedure as described in Example 1A was used. To a stirred ice-cooled solution of Boc-L-Glu-O-t-Bu (3.45 g, 11.4 mmol) in CH₂Cl₂ (120 mL) were successively added EDC (3.31 g, 17.3 mmol), HOBt (2.36 g, 17.5 mmol) and DIPEA (3.0 mL, 17.1 mmol). The resulting mixture was stirred at ice-cold temperature for another 25 min. A solution of H-D-Trp-OMe.HCl (2.20 g, 7.40 mmol) and DIPEA (3.0 mL, 17.1 mmol) in CH₂Cl₂ (40 mL) was then added. The resulting mixture was stirred at ice-cold temperature for 1 h, then allowed to warm to room temperature, and stirred for overnight.

After usual work-up as described in Example 1A, purification of the residue by column chromatography using a solvent gradient of a mixture of hexanes and EtOAc (8/2 and 6/4 ratio, v/v) as eluant afforded the title product (4.25 g, 74%) as a white foam. ¹H NMR (CDCl₃) δ ppm: 8.64 (s, 1H), 7.53 (d, J=7.7 Hz, 1H), 7.33 (d, J=8.0 Hz, 1H), 7.15 (t, J=7.2 Hz, 1H), 7.08 (t, J=7.5 Hz, 1H), 6.98 (s, 1H), 6.61 (d, J=7.2 Hz, 1H), 5.30 (d, J=7.8 Hz, 1H), 4.92 (q, J=6.8 Hz, 1H), 4.16-4.17 (m, 1H), 3.67 (s, 3H), 3.28-3.34 (m, 2H), 2.16-2.27 (m, 2H), 2.05-2.14 (m, 1H), 1.79-1.89 (m, 1H), 1.42-1.43 (m, 18H); ¹³C NMR (CDCl₃) δ ppm: 172.6 (C), 172.1 (C), 171.6 (C), 171.3 (C), 155.9 (C), 136.3 (C), 127.7 (C), 123.2 (CH), 122.2 (CH), 119.6 (CH), 118.6 (CH), 111.5 (CH), 109.9 (C), 82.3 (C), 79.9 (C), 53.7 (CH), 53.3 (CH), 52.4 (CH), 32.6 (CH₂), 28.9 (CH₂), 28.4 (CH₃), 28.1 (CH₃), 27.7 (CH₂); MS (m/z) 504 [M+1]⁺; Anal. Calcd for C₂₆H₃₇N₃O₇.0.25H₂O: C, 61.46; H, 7.44; N, 8.27. Found: C, 61.36; H, 7.50; N, 7.84

### B. Synthesis of Boc-L-iGlu-D-Trp-OH

To a stirred solution of Boc-L-Glu-((-D-Trp-OMe)-alpha-O-t-Bu (3.94 g, 7.82 mmol) in MeOH (50 mL) was added a solution of NaOH (654 mg, 16.4 mmol) in H₂O (20 mL). The resulting solution was stirred at room temperature overnight. 1N NaOH solution (150 mL) was added to the reaction mixture and the aqueous material was washed with EtOAc (3×100 mL). The aqueous layer was acidified with a 3N HCl solution to pH about 2, then extracted with EtOAc (3×100 mL). The organic fractions were combined, dried over Na₂SO₄ and concentrated under reduced pressure. Purification of the residue by column chromatography on silica gel using a solvent gradient of a mixture of CH₂Cl₂ and MeOH (85/15, 70/30 ratio, v/v) as eluant afforded the title product (0.55 g, 97%) as a pink foam.

¹H NMR (MeOD-D₄) δ ppm: 7.58 (d, J=7.7 Hz, 1H), 7.31 (d, J=8.0 Hz, 1H), 7.05-7.09 (m, 2H), 6.99 (t, J=7.3 Hz, 1H), 4.61-4.67 (m, 1H), 4.13 (br, 1H), 3.30-3.38 (m, 2H), 3.12-3.18 (m, 1H), 2.21-2.27 (m, 2H), 1.98-2.06 (m, 1H), 1.81-1.88 (m, 1H), 1.42 (s, 9H); ¹³C NMR (MeOD-d₄)*ppm: 158.2 (C), 138.1 (C), 129.1 (C), 124.5 (CH), 122.4 (CH), 119.9 (CH), 119.5 (CH), 112.3 (CH), 111.5 (C), 80.6 (C), 33.5 (CH₂), 28.9 (CH₃), 28.7 (CH₂); MS (m/z) 490 [M+1]⁺.

### C. Synthesis of H-L-iGlu-D-Trp-OH, mono ammonium salt (1:1)

HCl gas was bubbled into a stirred ice-cooled (about 0° C.) solution of Boc-L-iGlu-D-Trp-OH as described above (500 mg, 1.0 mmol) in a solvent mixture of CH₂Cl₂ (20 mL) and EtOAc (10 mL) for 30 min. The reaction mixture was stirred at ice-cold temperature for 1 h. The reaction was completed, as monitored by HPLC (column: Waters C18, 3.9×150 mm, WAT046980, mobile phase: solvent gradient of a mixture of 0.035% HClO₄ (pH=2.0-2.5) and acetonitrile, flow rate: 1 mL/min, 8: 210-270 nm).

The reaction mixture was evaporated to dryness to a dark purple foam. Purification of the residue by column chromatography using a solvent gradient of a mixture of isopropanol and ammonium hydroxide (28-30% NH₄OH) (85/15 and 70/30 ratio, v/v) as eluant afforded the title product (333 mg, 88%) as an orange thick oil. ¹H NMR (D₂O) δ ppm: 7.64 (d, J=7.9 Hz, 1H), 7.44 (d, J=8.0 Hz, 1H), 7.16-7.19 (m, 2H), 7.10 (t, J=7.5 Hz, 1H), 4.52 (q, J=3.7 Hz, 1H), 3.44 (t, J=6.3 Hz, 1H), 3.29-3.34 (m, 1H), 3.03-3.09 (m, 1H), 2.17-2.30 (m, 2H), 1.76-1.96 (m, 2H); ¹³C NMR (D₂O) δ ppm: 181.2 (C), 176.6 (C), 176.5 (C), 138.8 (C), 129.9 (C), 126.9 (CH), 124.5 (CH), 121.9 (CH), 121.4 (CH), 114.5 (CH), 113.1 (C), 58.5 (CH), 56.7 (CH), 51.6 (CH), 34.2 (CH₂), 30.2 (CH₂), 28.9 (CH₂); MS (m/z) 356 [Diacid+Na]⁺, 334 [Diacid+1]⁺, Anal. Calcd. for C₁₆H₂₂N₄O₅.2.35H₂O: C, 48.94; H, 6.85; N, 14.27. Found: C, 48.94; H, 6.64; N, 14.28.

### Example 12

### HPLC analysis of D-isoglutamyl-D-tryptophan, L-isoglutamyl-L-tryptophan, L-isoglutamyl-D-tryptophan and D-isoglutamyl-L-tryptophan

The diacid of the four diastereomers are analyzed on chiral column HPLC. The D, D- and L, D-diastereoisomers are from the examples above, while the D, L-isomer is from Bachem and the L, L-isomer is from Sigma. The analysis using chiral HPLC column (Table 1) shows that the D-isoglutamyl-D-tryptophan obtained is free from the other diastereomers, namely (D,L), (L, L) and (L, D) isomers.

TABLE 1
HPLC Analysis of H-iGlu-Trp-OH

H-iGlu-Trp-OH

| | Retention time | Retention time |
|---|---|---|
| | (HPLC Method A) | (HPLC Method B) |
| | (min.) | (min.) |
| (D, D) diastereomer | 19.32 | 3.92 |
| (D, L) diastereomer | 9.46 | 3.92 |
| (L, D) diastereomer | 13.99 | 3.87 |
| (L, L) diastereomer | 6.70 | 3.91 |

Method A:
Column: CHIROBIOTIC ® TAG 5 μM, 4.6 × 250 mm
Mobile phase: 20 mM ammonium acetate (pH = 4.1)/MeOH (80/20)
Flow rate: 0.8 mL/min
Detection λ: 222, 254, 282, 450 nm
Column Temperature: 45° C.
Method B:
Column: Symmetry C18, part no: WAT 046980
Mobile phase: HClO₄(pH = 2)/CH₃CN (85/15)
Flow rate: 1.0 mL/min
Detection λ: 210-280 nm

In method A, the samples are analyzed with a chiral column. The retention times of all four diastereomers are distinctly different. In method B, the samples are analyzed with a normal reverse phase column, there is virtually no difference in the retention times of the samples. The mono ammonium salt of D-isoglutamyl-D-tryptophan disclosed in the present invention is stable after 2 years of storage. HPLC analysis by method B showed that the purity at 254 nm is 99.8%.

### Example 13

### A. Preparation of H-D-Glu-(gamma-D-Trp-OMe)-alpha-OBzl HCl salt {(2R)-Amino-(4R)-[2-(1H-indol-3-yl)-1-methoxycarbonyl-ethylcarbamoyl]-butyric acid benzyl ester hydrochloride}

In a 250-mL 3 N round bottom flask equipped with a magnetic stir bar was placed Boc-D-Glu-(gamma-D-Trp-OMe)-alpha-OBzl (20 g, 0.037 mol) and 100 mL of dichloromethane to give a clear solution upon stirring. The solution was cooled by an ice-NaCl cooling bath to −10° C. HCl gas was bubbled into the cold solution. During the reaction the temperature was in the range of −4° C. to −10° C. The reaction was completed in about 1 hour. A white solid came out from the solution. The solid product was collected by filtration. The solid was washed with dichloromethane (40 mL×2), air-dried first, then dried in a vacuum oven at 42° C. overnight to give 16.4 g (94%, HPLC purity 98.2%). ¹H NMR (DMSO-d₆) δ ppm: 10.93 (s, 1H), 8.61 (b, 3H), 8.50 (d, J=7.4 Hz, 1H), 7.47 (d, J=7.4 Hz, 1H), 7.40-7.32 (m, 6H), 7.17 (s, 1H), 7.06 (t, J=7.4 Hz, 1H), 6.97 (t, J=7.4 Hz, 1H), 5.26-5.14 (m, 2H), 4.51-4.46 (m, 1H), 4.05-3.95 (m, 1H), 3.56 (s, 3H), 3.16-3.11 (m, 1H), 3.06-3.01 (m, 1H), 2.40-2.26 (m, 2H), 2.00-1.98 (m, 2H). HPLC method: Column: XTerra MS C18 5 μm 4.6×250 mm; Mobile phase: A=the aqueous phase: 4 mM Tris, 2 mM EDTA, pH 7.4, B=the organic phase: CH₃CN. The gradient program: B %: 0 min. 5%, 15 min. 55%, 30 min. 55%, 32 min. 5%, 40 min. 5%. Flow rate: 1 ml/min; injection volume=5 μL; wavelength: 222, 254, 282, 450 nm. Rₜ of the starting material=25.1 min; Rₜ of the product=17.2 min.

### B. Preparation of (2R)-Amino-(4R)-[2-(1H-indol-3-yl)-1-methoxycarbonyl-ethylcarbamoyl]-butyric acid methyl ester hydrochloride; H-D-Glu-(gamma-D-Trp-OMe)-alpha-OMe HCl salt

In a 250-mL 3 N round bottom flask equipped with a magnetic stir bar was placed Boc-D-Glu-(gamma-D-Trp-OMe)-alpha-OMe (2.8 g, 6.06 mmol) and methanol (30 mL) to give a clear solution upon stirring. The solution was cooled by an ice-NaCl cooling bath to −12° C. HCl gas was bubbled into the cold solution. During the reaction the temperature was in the range of −12° C. to +9° C. The reaction was completed in about 30 minutes. About half of the reaction mixture was concentrated to dryness to give a solid 1.3 g (HPLC purity 96.8%).

¹H NMR (DMSO-d₆) δ ppm: 10.90 (s, 1H), 8.48-8.46 (m, 4H), 7.48 (d, J=7.8 Hz, 1H), 7.34 (d, J=8.0 Hz, 1H), 7.16 (s, 1H), 7.07 (t, J=7.4 Hz, 1H), 6.98 (t, J=7.4 Hz, 1H), 4.52-4.47 (m, 1H), 4.05-3.99 (m, 1H), 3.69 (s, 3H), 3.58 (s, 3H), 3.17-3.12 (m, 1H), 3.07-3.01 (m, 1H), 2.37-2.23 (m, 2H), 1.98-1.91 (m, 2H). HPLC method: Column: XTerra MS C18 5 μm 4.6×250 mm; Mobile phase: A=the aqueous phase: 4 mM Tris, 2 mM EDTA, pH 7.4; B=the organic phase: CH₃CN. The gradient program: B %: 0 min. 5%, 15 min. 55%, 30 min. 55%, 32 min. 5%, 40 min. 5%. Flow rate: 1 ml/min. Injection volume=5 μL; wavelength: 222, 254, 282, 450 nm; Rₜ of the starting material=18.7 min, Rₜ of the product=13.0 min.

### Example 14

### Preparation of a solution of the base addition salt of D-isoglutamyl-D-tryptophan and its conversion to Thymodepressin D-isoglutamyl-D-tryptophan

### Procedure 14A:

The starting material H-D-Glu-(gamma-D-Trp-OMe)-alpha-OBzl HCl salt (4.0 g, 8.4 mmol) was placed in a 250 mL 3 N round bottom flask equipped with a magnetic stir bar. Methanol (20 mL) was added to give a clear solution. The solution was cooled by an ice-NaCl salt bath to −10° C. A NaOH solution (3 N, 8.4 mL, 25.2 mmol) was added. HPLC was used to monitor the reaction. After 2 hours the HPLC analysis of the reaction mixture indicated that the reaction was not completed yet. A NaOH solution (3 N, 1.4 mL, 4.2 mmol) was added. At this point, a total of 29.4 mmol of NaOH was added. After another 4 hours the HPLC analysis of the reaction mixture indicated that the product in the reaction mixture was higher than 95.2%. The reaction was stopped. The reaction mixture was acidified, under cooling using an ice-water bath, to pH 6.5 by adding hydrochloric acid (6 N, ˜1.3 mL, 7.8 mmol). The resulting solution was concentrated to remove most of methanol to a volume of 15 mL. The solution was washed with ethyl acetate (15 mL×2). The solution was filtered to collect the filtrate. The filtrate was further acidified to pH 3 by adding hydrochloric acid (6N, ˜1.3 mL, 7.8 mmol). At this point, a total of ˜15.6 mmol of hydrochloric acid was used. A solid formed upon stirring at room temperature. The mixture was stirred overnight. The solid was collected by filtration. The solid was air-dried to get a crude product 2.4 g. The solid then put back to a round bottom flask. Deionized water (15 mL) was added, and the mixture was stirred for 2 hours. The solid was collected by filtration. The solid was air-dried again, and then put back to a round bottom flask again. Deionized water (15 mL) was added, and the mixture was stirred for 1 hour. The solid was collected by filtration, and washed with ice-cold deionized water (6 mL×3). The solid was proven to be chloride free by silver nitrate test. The solid was air-dried, then put into the vacuum oven at 42° C. for 19 hours to give 1.3 g (46%, HPLC purity 98.8%). The filtrates and water washing solutions were combined and concentrated for the isolation of a second crop of product. ¹H NMR (D₂O—NaOD pH 7.0) δ ppm: 7.59 (d, J=7.6 Hz, 1H), 7.38 (d, J=7.6 Hz, 1H), 7.15-7.12 (m, 2H), 7.05 (t, J=7.2 Hz, 1H), 4.47-4.44 (m, 1H), 3.40 (t, J=6.1 Hz, 1H), 3.30-3.25 (m, 1H), 3.03-2.97 (m, 1H), 2.3-2.1 (m, 2H), 1.84-1.69 (m, 2H). MS (m/z) 334.3 [M+1]⁺. HPLC method: Column: XTerra MS C18 5 μm 4.6×250 mm. Mobile phase: A=the aqueous phase: 4 mM Tris, 2 mM EDTA, pH 7.4; B=the organic phase: CH₃CN. The gradient program: B %: 0 min. 5%, 15 min. 55%, 30 min. 55%, 32 min. 5%, 40 min. 5%. Flow Rate: 1 ml/min. Injection volume=5 μL. Wavelength: 222, 254, 282, 450 nm. Rₜ of the product=6.5 min.

### Procedure 14B:

Lithium hydroxide monohydrate (0.374 g, 8.9 mmol) was dissolved in 3.5 mL of deionized water. The solution was placed in a 100 mL 1 N round bottom flask equipped with a magnetic stir bar. 6.5 mL of methyl tert-butyl ether was added to the solution. At room temperature the starting material H-D-Glu-(gamma-D-Trp-OMe)-alpha-OBzl HCl salt (2.0 g, 4.2 mmol) was added to form a suspension. Methanol (2 mL) was added, most of the solid dissolved. HPLC was used to monitor the reaction. There was still starting material in the reaction mixture after stirring at room temperature overnight. Lithium hydroxide monohydrate (0.190 g, 4.5 mmol) was dissolved in 2 mL of deionized water, and added to the reaction mixture followed by addition of 2 mL of methanol. At this point, a total of 13.4 mmol of LiOH was added. After 4 hours the HPLC analysis of the reaction mixture indicated that the reaction was not completed yet. Lithium hydroxide monohydrate (0.100 g, 2.4 mmol) was dissolved in 1 mL of deionized water, and added to the reaction mixture. At this point, a total of 15.8 mmol of LiOH was added. After another 2.5 hours the HPLC analysis of the reaction mixture indicated that the product in the reaction mixture was higher than 97.5%. The reaction was stopped. The reaction mixture was poured into a separatory funnel and the 2 phases separated. The aqueous phase was washed with ethyl acetate (15 mL×2). The aqueous phase was acidified, under cooling using an ice-water bath, to pH 6 by adding hydrochloric acid (6 N, ˜650 μL, 3.9 mmol). The aqueous phase was concentrated to 5 mL, and filtered to collect the filtrate. The filtrate was further acidified to pH 3 by adding hydrochloric acid (6 N, ˜700 μL, 4.2 mmol). At this point, a total of ˜8.1 mmol of hydrochloric acid was used. A solid formed upon stirring at room temperature. The solid was collected by filtration. The solid was air-dried, then put back to a round bottom flask. Deionized water (6 mL) was added, and the mixture was stirred for 15 minutes. The solid was collected by filtration, and washed with ice-cold deionized water (6 mL×6). The solid was proven to be chloride free by silver nitrate test. The solid was air-dried, then put into the vacuum oven at 42° C. for 12 hours to give 0.44 g (31%, HPLC purity 98.5%). The filtrates and water washing solutions were combined and concentrated for further isolation of a second crop of the product.

¹H NMR (D₂O—NaOD pH 6.0) δ ppm: 7.59 (d, J=7.7 Hz, 1H), 7.38 (d, J=7.7 Hz, 1H), 7.15-7.12 (m, 2H), 7.05 (t, J=7.2 Hz, 1H), 4.47-4.44 (m, 1H), 3.41 (t, J=6.0 Hz, 1H), 3.29-3.25 (m, 1H), 3.03-2.97 (m, 1H), 2.3-2.1 (m, 2H), 1.83-1.58 (m, 2H).

HPLC method: Column: XTerra MS C18 5 μm 4.6×250 mm. Mobile phase: A=the aqueous phase: 4 mM Tris, 2 mM EDTA, pH 7.4; B=the organic phase: CH₃CN. The gradient program: B %: 0 min. 5%, 15 min. 55%, 30 min. 55%, 32 min. 5%, 40 min. 5%.

1 ml/min. Injection volume=5 μL. Wavelength: 222, 254, 282, 450 nm. Rₜ of the product=6.5 min.

Thymodepressin prepared in the present invention has a water solubility of from about 20 to about 23 mg per ml in water. The water washing in Procedures A & B serves to remove inorganic salts such as sodium chloride or lithium chloride. In large scale preparation, the volume of aqueous washing can be controlled by computing the amount of inorganic salt present and using the solubility to determine the amount of water required to wash the product.

### Example 15

### Preparation of ethyl (2R)-2-amino-5-{[(2R)-1-ethoxy-3-(1H-indol-3-yl)-1-oxopropan-2-yl]amino}-5-oxopentanoate or H-D-Glu(D-Trp-OEt)-OEt (Apo838)

### A. Preparation of Cbz-D-Glu(D-Trp-O-Et)-O-Et

H-D-Trp-O-Et hydrochloride (1.00 g, 3.72 mmol), Cbz-D-Glu-O-Et (1.15 g, 3.72 mmol), HOBt hydrate (0.57 g, 3.72 mmol) and EDCl (0.71 g, 3.72 mmol) were mixed together in dichloromethane (10 mL) at room temperature. Then DIPEA (0.48 g, 3.72 mmol) was added and the reaction mixture was stirred at room temperature for overnight. The reaction mixture was concentrated in vacuo by rotary evaporation to remove volatile materials. The residue was diluted with ethyl acetate. The resulting organic suspension was successively washed with water, saturated sodium bicarbonate, water, 1N HCl, water and brine. After drying over MgSO₄, the organic layer was concentrated and the residue was triturated with a mixture of ether-hexanes to give Cbz-D-Glu(D-Trp-O-Et)-O-Et (1.25 g) as a white solid. Yield=64%; ¹H NMR (DMSO-D₆, 400 MHz) δ (ppm): 8.04 (br. s, 1H), 7.52 (d, J=7.1 Hz, 1H), 7.28-7.40 (m, 6H), 7.18 (t, J=7.6 Hz, 1H), 7.06-7.14 (m, 1H), 7.00 (s, 1H), 6.17 (d, J=7.1 Hz, 1H), 5.59 (d, J=7.1 Hz, 1H), 5.10 (s, 2H), 4.85-4.97 (m, 1H), 4.22-4.35 (m, 1H), 4.06-4.21 (m, 4H), 3.22-3.39 (m, 2H), 2.12-2.32 (m, 3H), 1.85-1.99 (m, 1H), 1.15-1.35 (m, 6H). MS-ESI (m/z) 524 [M+1]⁺.

### B. Preparation of H-D-Glu(D-Trp-O-Et)-O-Et (Apo838)

Cbz-D-Glu(D-Trp-O-Et)-O-Et 1.24 g (2.37 mmol) was mixed with 10% Pd/C (wet, 300 mg) in ethanol (20 mL) at room temperature. The reaction mixture was stirred under a H₂ filled balloon for an hour, then filtered through Celite™. The filtrate was concentrated to dryness by rotary evaporation and the residue was triturated with a mixture of ether-hexanes to give H-D-Glu(D-Trp-O-Et)-O-Et (Apo838, 0.72 g) as a white solid. Yield=78%. ¹H NMR (DMSO-D₆, 400 MHz) δ (ppm): 10.90 (s, 1H), 8.31 (d, J=7.3 Hz, 1H), 7.51 (d, J=7.8 Hz, 1H), 7.36 (d, J=8.0 Hz, 1H), 7.17 (br. s, 1H), 7.09 (t, J=7.3 Hz, 1H), 7.00 (t, J=7.5 Hz, 1H), 4.50 (q, J=6.9 Hz, 1H), 4.09 (q, J=7.0 Hz, 2H), 4.03 (q, J=7.0 Hz, 2H), 3.03-3.25 (m, 3H), 2.45-3.45 (br. above baseline hump), 2.23 (t, J=7.6 Hz, 2H), 1.75-1.82 (m, 1H), 1.53-1.62 (m, 1H), 1.19 (t, J=7.1 Hz, 3H), 1.09 (t, J=7.0 Hz, 3H); MS-ESI (m/z): 390 [M+1]⁺.

### Example 16

### Preparation of methyl (2R)-2-amino-5-{[(2R)-3-(1H-indol-3-yl)-1-methoxy-1-oxopropan-2-yl]amino}-5-oxopentanoate hydrochloride or H-D-Glu(D-Trp-OMe)-OMe.HCl

To an ice-cooled suspension of D-gamma-glutamyl-D-tryptophan (Apo805, 5.0 g, 15 mmol) in methanol (60 mL) was bubbled HCl gas over several time periods. Each time after bubbling HCl gas for a few minutes the reaction mixture was stirred at ice-cold temperature and the progress of the reaction was monitored by HPLC. Column: XTerra MS, C18, 5 μm, 4.6×250 mm; Mobile phase: A=the aqueous phase: 4 mM Tris, 2 mM EDTA, pH 7.4; B=the organic phase: CH₃CN; Flow rate=1 mL/min; injection volume=5 μL; λ: 222, 254, 280, 450 nm; Method: Time in min-B %: 0-5%, 15-55%, 25-55%, 25.05-5%, 30-5%;: RT of starting material=5.48 min; RT of product=12.83 min. The starting material was converted to the title compound within 4 h. Nitrogen gas was bubbled into the reaction mixture, and the mixture was then evaporated to dryness in vacuo to give the title compound as a light pink solid. The solid was washed with ethyl ether and dried in a vacuum oven at 35° C. for overnight to give the titled compound H-D-Glu(D-Trp-OMe)-OMe.HCl. Yield=91% (5.4 g); HPLC (AUC) purity at 280 nm=95.1%; ¹H NMR (DMSO-D₆) δ ppm: 10.93 (s, 1H), 8.65-8.55 (br, 3H), 8.48 (d, J=7.3 Hz, 1H), 7.48 (d, J=7.8 Hz, 1H), 7.34 (d, J=8.0 Hz, 1H), 7.17 (s, 1H), 7.07 (t, J=7.4 Hz, 1H), 6.99 (t, J=7.4 Hz, 1H), 4.47-4.52 (m, 1H), 3.94-3.98 (m, 1H), 3.69 (s, 3H), 3.58 (s, 3H), 3.12-3.17 (m, 1H), 3.01-3.07 (m, 1H), 2.23-2.37 (m, 2H), 1.91-1.98 (m, 2H). MS-ESI (m/z): 362 [M-HCl+1]⁺ (free base).

### Example 17

### Preparation of propan-2-yl (2R)-2-amino-5-{[(2R)-3-(1H-indol-3-yl)-1-oxo-1-(propan-2-yloxy)propan-2-yl]amino}-5-oxopentanoate hydrochloride or H-D-Glu(D-Trp-O-i-Pr)-O-i-Pr.HCl (Apo845.HCl)

To a solution of N-(tert-butoxycarbonyl)-D-gamma-glutamyl-D-tryptophan (Apo806, 5.00 g, 11.5 mmol) in DMF (60 mL) was added potassium carbonate (7.97 g, 57.7 mmol) followed by 2-iodopropane (2.70 mL, 26.5 mmol). The mixture was stirred at RT for overnight.

The reaction mixture was quenched with water and then extracted with EtOAc. The combined EtOAc layers was washed with brine, dried over anhydrous Na₂SO₄ and filtered. The filtrate was concentrated to a volume of about 150 mL and cooled in an ice/water bath. Then, HCl gas was bubbled into the solution for about 0.5 h. The reaction mixture was evaporated to dryness. The residue was pre-purified by flash column chromatography on silica gel, and further purified by Biotage with reverse C-18 column. The product obtained was stirred in a solution of 2M HCl in Et₂O (10 mL), and then concentrated to dryness in vacuo. The residue was dried under vacuum to afford the title compound (1.24 g) as off-white solid. Yield=20.1%; ¹H NMR (DMSO-D₆, 400 MHz) δ ppm: 10.93 (br. s, 1H), 8.55 (br. s, 3H), 8.48 (d, J=7.1 Hz, 1H), 7.49 (d, J=8.1 Hz, 1H), 7.34 (d, J=8.1 Hz, 1H), 7.17 (s, 1H), 7.07 (t, J=7.6 Hz, 1H), 6.94-7.03 (m, 1H), 4.99 (d of t, J=12.1, 6.1 Hz, 1H), 4.81 (d of t, J=12.4, 6.4 Hz, 1H), 4.38-4.50 (m, 1H), 3.91 (t, J=6.6 Hz, 1H), 2.97-3.19 (m, 2H), 2.22-2.42 (m, 2H), 1.96 (q, J=7.1 Hz, 2H), 1.21-1.24 (m, 6H), 1.14 (d, J=6.1 Hz, 3H), 1.01 (d, J=6.1 Hz, 3H); MS-ESI (m/z): 418 [M+1]⁺ (free base).

### Example 18

### Preparation of H-D-Glu(D-Trp-O-Me)-O-Et (Apo916)

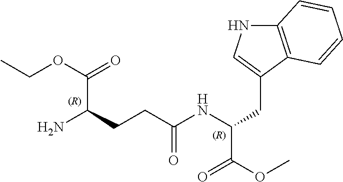

### A. Preparation of Cbz-D-Glu(D-Trp-OH)—O-Et

Cbz-D-Glu-O-Et (27.3 g, 88.3 mmol), HOSu (10.1 g, 88.3 mmol) and EDCl hydrochloride (16.9 g, 88.3 mmol) were mixed in DMF (200 mL) and then stirred at RT for overnight. To the reaction mixture was added D-Trp-OH (18.0 g, 88.3 mmol). After stirring at RT for another 6 h, the mixture was quenched with a 0.6 N HCl solution (300 mL). The mixture was extracted with ethyl acetate (2×). The organic layers were combined and washed with water (3×) and brine (100 mL), dried over MgSO₄ and filtered. The filtrate was concentrated to dryness by rotary evaporation and the resulting solid was triturated with ether to give Cbz-D-Glu(D-Trp-OH)—O-Et (26.4 g) as a white solid. Yield=60%; ¹H NMR (DMSO-D₆, 400 MHz) δ (ppm): 12.60 (br. s, 1H), 10.83 (br. s, 1H), 8.13 (d, J=7.1 Hz, 1H), 7.73 (d, J=8.1 Hz, 1H), 7.51 (d, J=7.1 Hz, 1H), 7.21-7.44 (m, 6H), 7.12 (s, 1H), 7.05 (t, J=7.1 Hz, 1H), 6.88-7.02 (m, 1H), 4.91-5.22 (m, 2H), 4.36-4.58 (m, 1H), 3.84-4.21 (m, 3H), 3.14 (dd, J=14.1, 5.1 Hz, 1H), 2.99 (dd, J=14.7, 8.6 Hz, 1H), 2.08-2.31 (m, 2H), 1.80-1.98 (m, 1H), 1.57-1.80 (m, 1H), 1.07-1.24 (m, 3H); MS-ESI (m/z): 496 [M+1]⁺.

### B. Preparation of Cbz-D-Glu(D-Trp-O-Me)-O-Et

A mixture of Cbz-D-Glu(D-Trp-OH)—O-Et (1.00 g, 2.00 mmol), iodomethane (0.57 g, 4.04 mmol) and potassium carbonate (0.56 g, 4.04 mmol) in DMF (8 mL) was stirred for overnight. The reaction mixture was diluted with water, extracted with ethyl acetate. The organic layer was washed with water (4×), then with brine, dried over MgSO₄ and filtered. The filtrate was concentrated to dryness in vacuo and the residue was triturated with a mixture of ether-hexanes to give Cbz-D-Glu(D-Trp-OMe)-OEt (0.77 g). Yield=74%; ¹H NMR (CDCl₃, 400 MHz) (ppm): 8.03 (br. s, 1H), 7.51 (d, J=7.1 Hz, 1H), 7.28-7.40 (m, 6H), 7.18 (t, J=7.6 Hz, 1H), 7.06-7.14 (m, 1H), 7.00 (s, 1H), 6.17 (d, J=8.1 Hz, 1H), 5.57 (d, J=7.1 Hz, 1H), 5.10 (s, 2H), 4.89-4.99 (m, 1H), 4.24-4.33 (m, 1H), 4.17 (q, J=7.1 Hz, 2H), 3.69 (s, 3H), 3.23-3.42 (m, 2H), 2.13-2.33 (m, 3H), 1.87-2.00 (m, 1H), 1.25 (t, J=7.1 Hz, 3H); MS-ESI (m/z): 510 [M+1]⁺.

### C. Preparation of H-D-Glu(D-Trp-O-Me)-O-Et (Apo916)

Cbz-D-Glu(D-Trp-O-Me)-O-Et (600 mg, 1.17 mmol) was mixed with 10% Pd—C (wet, 200 mg) in ethyl acetate (20 mL). The reaction mixture was stirred at room temperature under a hydrogen atmosphere using a balloon for overnight. After filtration through Celite™, the filtrate was concentrated to dryness by rotary evaporation. The residue was then triturated with a mixture of ether-hexanes to give H-D-Glu(D-Trp-O-Me)-O-Et (420 mg). Yield=95%. ¹H NMR (CDCl₃, 400 MHz) δ (ppm): 8.25 (br. s, 1H), 7.54 (d, J=8.1 Hz, 1H), 7.35 (d, J=8.1 Hz, 1H), 7.19 (t, J=7.6 Hz, 1H), 7.07-7.14 (m, 1H), 7.01 (s, 1H), 6.46 (d, J=8.1 Hz, 1H), 4.89-5.03 (m, 1H), 4.13 (q, J=7.1 Hz, 2H), 3.70 (s, 3H), 3.17-3.41 (m, 3H), 2.18-2.40 (m, 2H), 1.98-2.13 (m, 1H), 1.67-1.80 (m, 1H), 1.60 (br. s, 2H), 1.24 (t, J=7.1 Hz, 3H); MS-ESI (m/z): 376 [M+1]⁺.

### Example 19

### Preparation of H-D-Glu(D-Trp-O-i-Pr)-O-Et hydrochloride salt (915.HCl)

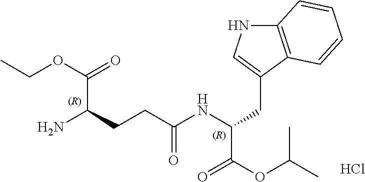

### A. Preparation of Cbz-D-Glu(D-Trp-O-i-Pr)-O-Et

Proceeding in a similar manner as described in Example 18B above, Cbz-D-Glu(D-Trp-O-i-Pr)-OEt (0.85 g, Yield=78%) was prepared from the reaction of Cbz-D-Glu(D-Trp-OH)—O-Et (1.00 g, 2.0 mmol) with 2-iodopropane (0.69 g, 4.04 mmol) and potassium carbonate (0.56 g, 4.04 mmol) in DMF (8 mL) at RT for overnight. ¹H NMR (CDCl₃, 400 MHz) δ (ppm): 8.04 (br. s, 1H), 7.54 (d, J=7.1 Hz, 1H), 7.28-7.41 (m, 6H), 7.18 (t, J=7.6 Hz, 1H), 7.05-7.14 (m, 1H), 7.00 (s, 1H), 6.15 (d, J=7.1 Hz, 1H), 5.59 (d, J=8.1 Hz, 1H), 5.10 (s, 2H), 4.98 (dt, J=12.4, 6.4 Hz, 1H), 4.81-4.92 (m, 1H), 4.22-4.33 (m, 1H), 4.08-4.22 (m, 2H), 3.20-3.41 (m, 2H), 2.11-2.29 (m, 3H), 1.92 (dq, J=14.1, 7.1 Hz, 1H), 1.12-1.33 (m, 9H); MS-ESI (m/z): 538 [M+1]⁺.

### B. Preparation of H-D-Glu(D-Trp-O-i-Pr)-O-Et hydrochloride (Apo915.HCl)

Proceeding in a similar manner as described in Example 18C above, hydrogenation of Cbz-D-Glu(D-Trp-O-i-Pr)-O-Et (500 mg, 0.93 mmol) with 10% Pd—C (wet, 160 mg) in ethyl acetate (20 mL) under a hydrogen atmosphere using a balloon for overnight afforded H-D-Glu(D-Trp-O-i-Pr)-O-Et. The latter was converted to its hydrochloride salt (Apo915.HCl, 245 mg, yield=61%) with 4M HCl in dioxane (0.25 mL). ¹H NMR (DMSO-D₆, 400 MHz) δ (ppm): 10.91 (br. s, 1H), 8.37-8.61 (m, 4H), 7.48 (d, J=7.1 Hz, 1H), 7.33 (d, J=8.1 Hz, 1H), 7.16 (s, 1H), 7.06 (t, J=7.6 Hz, 1H), 6.94-7.01 (m, 1H), 4.80 (dt, J=12.4, 6.4 Hz, 1H), 4.34-4.51 (m, 1H), 4.08-4.23 (m, 2H), 3.95 (t, J=6.1 Hz, 1H), 3.08-3.20 (m, 1H), 2.94-3.07 (m, 1H), 2.21-2.39 (m, 2H), 1.88-2.04 (m, 2H), 1.20 (t, J=7.1 Hz, 3H), 1.13 (d, J=6.1 Hz, 3H), 0.99 (d, J=6.1 Hz, 3H); MS-ESI (m/z): 404 [M+1]⁺ (free base).

### Example 20

### Preparation of D-Glu(D-Trp-O-t-Bu)-O-t-Bu (Apo920)

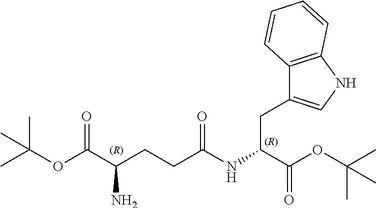

### A. Preparation of Cbz-D-Glu(D-Trp-O-t-Bu)-O-t-Bu

A. To an ice-water cooled suspension of Cbz-D-Glu(D-Trp) (25.0 g, 53.5 mmol) in tert-butyl acetate (230 mL) was added concentrated H₂SO₄ (7 mL) dropwise. After addition, the reaction mixture was sealed with a septum cap, and then stirred at RT for about 2.5 h. The reaction mixture was quenched with water. The separated organic layer was successively washed with water, an aqueous NaHCO₃ solution and brine, and then dried over anhydrous Na₂SO₄ and filtered. The filtrate was concentrated to dryness in vacuo. The residue was purified by flash column chromatography on silica gel using a mixture of EtOAc and hexanes (gradient, ratio from 1/4 to 1/1, v/v) as eluent, thereby affording Cbz-D-Glu(D-Trp-O-t-Bu)-O-t-Bu (3.93 g). Y=13%; ¹H NMR (DMSO-D₆, 400 MHz) δ (ppm): 10.85 (s, 1H), 8.19 (d, J=7.1 Hz, 1H), 7.63 (d, J=8.1 Hz, 1H), 7.51 (d, J=8.1 Hz, 1H), 7.24-7.43 (m, 6H), 7.13 (br. s, 1H), 7.07 (t, J=7.6 Hz, 1H), 6.94-7.04 (m, 1H), 4.95-5.13 (m, 2H), 4.35-4.48 (m, 1H), 3.83-3.95 (m, 1H), 3.05-3.17 (m, 1H), 2.92-3.05 (m, 1H), 2.10-2.32 (m, 2H), 1.82-1.96 (m, 1H), 1.66-1.81 (m, 1H), 1.39 (s, 9H), 1.28 (s, 9H); MS-ESI (m/z): 580 [M+1]⁺.

### B. Preparation of H-D-Glu(D-Trp-O-t-Bu)-O-t-Bu (Apo920)

A mixture of Cbz-D-Glu(D-Trp-O-t-Bu)-O-t-Bu (3.9 g, 6.7 mmol), and 10% Pd/C (wet, 1.5 g) in EtOH (150 mL) was hydrogenated under 40 psi hydrogen atmosphere in a Parr apparatus for 1.5 h. The reaction mixture was filtered, and the filtrate was concentrated to dryness to give a crude product (2.83 g). About 2 g of the crude was purified by flash column chromatography on silica gel using a mixture of CH₂Cl₂ and i-PrOH (gradient, ratio from 92/8 to 85/15, v/v) as eluent, thereby affording H-D-Glu(D-Trp-O-t-Bu)-O-t-Bu (Apo920, 1.52 g) as a white foamy solid. ¹H NMR (DMSO-D₆+D₂O, 400 MHz) δ (ppm): 7.49 (d, J=7.1 Hz, 1H), 7.33 (d, J=7.1 Hz, 1H), 7.12 (s, 1H), 7.06 (t, J=7.6 Hz, 1H), 6.95-7.03 (m, 1H), 4.34-4.42 (m, 1H), 3.03-3.17 (m, 2H), 2.90-3.03 (m, 1H), 2.15 (t, J=7.6 Hz, 2H), 1.64-1.80 (m, 1H), 1.46-1.63 (m, 1H), 1.37 (s, 9H), 1.27 (s, 9H); MS-ESI (m/z): 446 [M+1]⁺.

### Example 21

### Preparation of D-Glu(D-Trp-O-i-Pr)-O-t-Bu hydrochloride salt (Apo919.HCl)

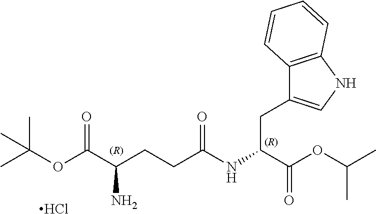

### A. Preparation of Boc-D-Glu(D-Trp-O-i-Pr)-O-t-Bu

To a solution of Boc-D-Glu-O-t-Bu (2.0 g, 6.6 mmol) in DMF (15 mL) was added N-hydroxysuccinimide (0.84 g, 7.3 mmol), followed by EDCl.HCl (1.39 g, 7.3 mmol), DIPEA (1.3 mL, 7.3 mmol). After stirring for 15 min, H-D-Trp-O-i-Pr.HCl (2.1 g, 7.3 mmol) was added followed by DIPEA (1.3 mL, 7.3 mmol). The resulting mixture was then stirred for overnight. The reaction mixture was quenched with water and then extracted with EtOAc. The EtOAc layer was successively washed with a 5% citric acid solution, brine, an aqueous NaHCO₃ solution and brine, then dried over anhydrous Na₂SO₄, filtered and concentrated to dryness. The crude product was purified by flash column chromatography on silica gel using a mixture of EtOAc and hexanes (1/1 ratio, v/v) as eluent, thereby affording Boc-D-Glu(D-Trp-O-i-Pr)-O-t-Bu (1.79 g, Y=51%) as a white foam solid. ¹H NMR (DMSO-D₆, 400 MHz) δ (ppm): 10.85 (br. s, 1H), 8.25 (d, J=7.1 Hz, 1H), 7.49 (d, J=8.1 Hz, 1H), 7.34 (d, J=8.1 Hz, 1H), 7.10-7.17 (m, 2H), 7.03-7.10 (m, 1H), 6.95-7.02 (m, 1H), 4.74-4.86 (m, 1H), 4.41 (q, J=7.1 Hz, 1H), 3.72-3.82 (m, 1H), 3.06-3.15 (m, 1H), 2.96-3.06 (m, 1H), 2.08-2.26 (m, 2H), 1.76-1.92 (m, 1H), 1.63-1.76 (m, 1H), 1.39 (s, 18H), 1.13 (d, J=6.1 Hz, 3H), 0.99 (d, J=6.2 Hz, 3H); MS-ESI (m/z): 532 [M+1]⁺.

### B. Preparation of H-D-Glu(D-Trp-O-i-Pr)-O-t-Bu hydrochloride salt (Apo920.HCl)

To an ice-water cooled solution of 4M HCl in dioxane (5.5 mL) was added Boc-D-Glu(D-Trp-O-i-Pr)-O-t-Bu (1.43 g, 2.7 mmol) in one portion. The reaction mixture was allowed to warm to RT, stirred for another 30 min, then concentrated to dryness in vacuo. The residue was taken up in EtOAc, and then washed with an aqueous NaHCO₃ solution and followed by brine. The organic layer was dried over anhydrous Na₂SO₄, filtered and concentrated to dryness. The residue was dissolved in Et₂O, and a 2M HCl in Et₂O solution (0.8 mL) was then added. The mixture was evaporated to dryness and dried under vacuum to afford H-D-Glu(D-Trp-O-i-Pr)-O-t-Bu hydrochloride salt (Apo920.HCl, 0.63 g). Y=50%; ¹H NMR (DMSO-D₆, 400 MHz) δ (ppm): 10.91 (s, 1H), 8.48 (d, J=7.1 Hz, 1H), 8.39 (br. s, 3H), 7.50 (d, J=8.1 Hz, 1H), 7.34 (d, J=8.1 Hz, 1H), 7.16 (d, J=2.0 Hz, 1H), 7.07 (t, J=7.6 Hz, 1H), 6.94-7.03 (m, 1H), 4.81 (quin, J=6.3 Hz, 1H), 4.44 (q, J=7.1 Hz, 1H), 3.86 (t, J=6.1 Hz, 1H), 3.09-3.18 (m, 1H), 2.97-3.09 (m, 1H), 2.21-2.40 (m, 2H), 1.85-2.03 (m, 2H), 1.45 (s, 9H), 1.14 (d, J=6.1 Hz, 3H), 1.00 (d, J=6.3 Hz, 3H). MS-ESI (m/z): 432 [M+1]⁺.

### Example 22

### Preparation of H-D-Glu(D-Trp-O-ethyl)-O-benzyl hydrochloride (Apo925.HCl)

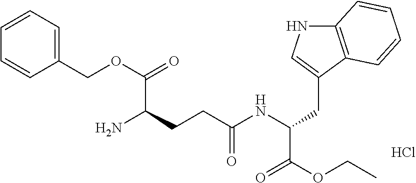

### A. Preparation of Boc-D-Glu(D-Trp-O-Et)-O-Bzl

Proceeding in a similar manner as described in Example 19A above, Boc-D-Glu(D-Trp-O-Et)-O-Bzl (2.90 g, yield=70%) was prepared from the reaction of H-D-Trp-O-Et hydrochloride (2.00 g, 7.4 mmol), EDCl.HCl (1.71 g, 8.9 mmol), HOBt hydrate (1.14 g, 7.4 mmol), Et₃N(2.63 g, 26.0 mmol) and Boc-D-Glu(OH)—O-Bzl (2.51 g, 7.4 mmol) in DMF (25 mL) at room temperature. ¹H NMR (DMSO-D₆, 400 MHz) δ (ppm): 10.85 (br. s, 1H), 8.27 (d, J=7.1 Hz, 1H), 7.48 (d, J=8.1 Hz, 1H), 7.26-7.42 (m, 7H), 7.14 (s, 1H), 7.07 (t, J=7.6 Hz, 1H), 6.98 (t, J=7.1 Hz, 1H), 5.04-5.19 (m, 2H), 4.40-4.52 (m, 1H), 3.99 (q, J=7.1 Hz, 3H), 2.97-3.17 (m, 2H), 2.11-2.31 (m, 2H), 1.84-1.98 (m, 1H), 1.65-1.84 (m, 1H), 1.38 (s, 8H), 1.26 (br. s, 1H), 1.05 (t, J=7.1 Hz, 3H); MS-ESI (m/z): 552 [M+1]⁺.

### B. Preparation of H-D-Glu(D-Trp-O-Et)-O-Bzl hydrochloride (Apo925.HCl)

Proceeding In a similar manner as described under example 19B, H-D-Glu(D-Trp-OEt)-O-Bzl hydrochloride (0.36 g, yield=45%) was obtained from the deprotection of Boc-D-Glu(D-Trp-O-Et)-O-Bzl (0.9 g, 1.63 mmol) with a 2M HCl in ether solution (10 mL). ¹H NMR (DMSO-D₆, 400 MHz) δ (ppm): 10.93 (br. s, 1H), 8.59 (br. s, 3H), 8.49 (d, J=7.1 Hz, 1H), 7.48 (d, J=8.1 Hz, 1H), 7.30-7.45 (m, 6H), 7.17 (s, 1H), 7.02-7.10 (m, 1H), 6.92-7.02 (m, 1H), 5.25 (d, J=12.1 Hz, 1H), 5.17 (d, J=12.1 Hz, 1H), 4.42-4.53 (m, 1H), 3.93-4.10 (m, 3H), 2.98-3.19 (m, 2H), 2.25-2.43 (m, 2H), 1.90-2.07 (m, 2H), 1.01-1.13 (m, 3H); MS-ESI (m/z): 452 [M+1]⁺ (free base).

### Example 23

### Preparation of H-D-Glu(D-Trp-O-Bzl)—O-Bzl(Apo926)

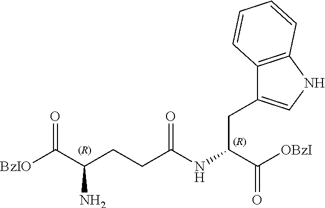

### A. Preparation of Boc-D-Glu(D-Trp-O-Bzl)-O-Bzl

To a suspension of Boc-D-Glu(D-Trp-OH)—OH (4.0 g, 9.2 mmol) and K₂CO₃ (5.1 g, 36.9 mmol) in DMF (60 mL) was added benzyl bromide (3.1 mL, 25.8 mmol) dropwise. The resulting suspension was then stirred at RT for overnight. The reaction mixture was quenched with water, and then extracted with EtOAc. The organic layer was washed with brine, then dried over anhydrous Na₂SO₄, filtered and the filtrate was evaporated to dryness. The residue was triturated with hexanes, and the precipitated solid was collected via suction filtration. Thus, Boc-D-Glu(D-Trp-O-Bzl)-O-Bzl (5.7 g, yield=100%) was obtained. ¹H NMR (DMSO-d₆, 400 MHz) δ (ppm): 10.87 (s, 1H), 8.36 (d, J=7.1 Hz, 1H), 7.49 (d, J=8.1 Hz, 1H), 7.24-7.42 (m, 10H), 7.04-7.21 (m, 4H), 6.98 (t, J=7.1 Hz, 1H), 5.07-5.18 (m, 2H), 4.95-5.07 (m, 2H), 4.46-4.61 (m, 1H), 3.96-4.07 (m, 1H), 3.11-3.21 (m, 1H), 3.01-3.11 (m, 1H), 2.16-2.31 (m, 2H), 1.85-2.03 (m, 1H), 1.67-1.83 (m, 1H), 1.38 (s, 9H); MS-ESI (m/z): 614[M+1]⁺.

### B. Preparation of H-D-Glu(D-Trp-O-Bzl)-O-Bzl (Apo926)

To a solution of Boc-D-Glu(D-Trp-OBn)-OBn (5.6 g, 9.1 mmol) in CH₂Cl₂ (50 mL) was added a 2M HCl in Et₂O solution (10 mL), and the resulting mixture was stirred at RT for overnight. The reaction mixture was evaporated to dryness to afford 5.04 g of a crude product. A portion of the crude material (4.33 g) was dissolved in water, and then extracted with EtOAc. The organic layer was washed with sodium bicarbonate aqueous, brine, then dried over anhydrous Na₂SO₄, filtered and the filtrate was concentrated to dryness under reduced pressure. The residue was purified by flash column chromatography on silica gel using a solvent mixture of i-PrOH and CH₂Cl₂ (gradient ratio from 5/95 to 10/90, v/v) as eluent, thereby affording H-D-Glu(D-Trp-O-Bzl)-O-Bzl (3.4 g) as a white solid. Yield=78%; ¹H NMR (DMSO-d₆+D₂O, 400 MHz) δ (ppm): 7.46 (d, J=8.1 Hz, 1H), 7.22-7.40 (m, 9H), 7.03-7.18 (m, 4H), 6.92-7.02 (m, 1H), 5.07 (s, 2H), 4.92-5.03 (m, 2H), 4.51 (t, J=7.1 Hz, 1H), 3.20-3.29 (m, 1H), 3.09-3.19 (m, 1H), 2.98-3.09 (m, 1H), 2.10-2.23 (m, 2H), 1.72-1.86 (m, 1H), 1.53-1.62 (m, 1H); MS-ESI (m/z): 514[M+1]⁺.

### Example 24

### Pharmacokinetic Studies of H-D-Glu-(gamma-D-Trp-OR²)-alpha-OR¹ or H-D-Glu(D-Trp-OR²)—OR¹ in Rats

General Procedure for Animal dosing

Groups of five male Sprague-Dawley rats weighing 250 to 300 g were utilized per dosing group. One day prior to dosing, venous and arterial catheters (made of 20 cm long polyurethane coiled tubing, and filled with 100 units/mL heparinized saline) were implanted into the jugular vein and carotid artery of each rat. Rats were fasted overnight prior to oral dosing and fed approximately 2 hours post-dosing. All dosing and blood sampling was performed on fully conscious rats. Tested compounds were administered either by oral gavage as solutions in water, or by intravenous injection (Apo805K1 only) as solution in 0.9% sodium chloride, final pH 7.0, at doses equivalent to 5 mg/kg (per Apo805 content). Blood (0.3 mL) was sampled from each animal from the carotid artery for up to 30 hours post-dosing, each sampling followed by an equivalent naive-blood replacement. The blood sample was immediately centrifuged (4300×g for 5 minutes at 4° C.), and frozen at −80° C. until LC/MS/MS analysis.

### General Procedure for LC-MS/MS Analysis of Plasma Drug Concentration

Metanol (200 μL) was added to plasma samples (50 μL) to precipitate plasma proteins. After brief vortexing and centrifugation, the supernatant (200 uL) was removed and dried at 40° C. under a stream if nitrogen. The sample was reconstituted in water (300 μL) and 25 μL was injected for analysis.

A Sciex API 365 LC/MS/MS spectrophotometer equipped with Ionics EP10+ and HSID, was used. A chiral column (Supelco-Astec CHIROBIOTIC™ TAG), 100×2.1 mm, 5 μm was used at ambient temperature. The mobile phase consisted of 0.1% formic acid in water (A) and 0.1% formic acid in acetonitrile (B) in a ratio of 88:12(A:B; v/v) and the flow rate was 0.6 mL/min. Positive ion electrospray ionization (ESI+) in MRM mode was used for analysis. Samples were analysed for the concentration of Apo805 (H-D-Glu(D-Trp-OH)—OH).

### Oral Bioavailability of Apo838 and Apo804 in Rats

Absolute oral bioavailability of pro-drugs Apo804 (R¹=benzyl, R²=methyl), and Apo838 (R¹═R²=ethyl) was compared to that of Apo805K1 (potassium salt of H-D-Glu(D-Trp-OH)—OH) in male Sprague-Dawley rats. Adult animals, five per group, were dosed orally with 5 mg/kg Apo805K1, Apo804, or Apo838 and intravenously with 5 mg/kg Apo805K1. As Apo804, and Apo838 are instantaneously converted to Apo805 in rat blood, only levels of Apo805 were measured in plasma collected at various time intervals post-dosing.

### PK Analysis

Non-compartmental analysis was performed using WinNonlin 5.2 software, on individual animal data. Bioavailability was calculated as a ratio of AUC_{INF}_{_{—}}D after oral dosing of test compound to AUC_{INF}_{_{—}}D after IV dosing of Apo805K1.

FIG. 1 shows the plasma concentration of Apo805 after oral dosing of Apo804 or Apo805K1. FIG. 2 shows the plasma concentration of Apo805 after oral dosing of Apo838 or Apo805K1. Absolute oral bioavailability, calculated as a ratio of the area under the time-plasma concentration curve (AUC) after oral dosing to AUC after intravenous dosing was 46% for Apo804, and 55% for Apo838. Absolute bioavailability of Apo805K1 was only 12%. Thus, the bioavailability of pro-drugs is significantly enhanced compared to Apo805K1.

### Example 25

### Biotransformation Studies of H-D-Glu-(gamma-D-Trp-OR²)-alpha-OR¹ or H-D-Glu(D-Trp-OR²)—OR¹ in Rats in Human Hepatocytes

### General Procedure:

LiverPool® cryopreserved human hepatocytes (pooled from 10 male donors) was obtained from Celsis In Vitro Technologies. The hepatocytes were stored in liquid nitrogen until used. Just before the assay, the hepatocytes were quickly thawed at 37° C. and centrifuged at 100×g for 10 min. The media was removed and cells were re-suspended in PBS at a density of 4×10⁶ cells/mL. The compound H-D-Glu-(gamma-D-Trp-OR²)-alpha-OR¹ (100 μM) were incubated with 0.1×10⁶ hepatocytes in 50 μL volume. After 10, 20, 60, 120 and 240 min of incubation, the reaction was quenched by adding an equal volume of 5% (w/v) TCA. The “time 0” sample was generated by adding TCA before the test compound. After brief vortexing and 10-min incubation on ice, samples were centrifuged (16,000×g, 10 min) and the supernatants were analyzed by HPLC with UV detection.

### HPLC Analysis of Pro-Drugs in Plasma and Hepatocytes Samples:

HPLC analysis was done using an Agilent 1100 series HPLC system consisting of a programmable multi-channel pump, auto-injector, vacuum degasser and HP detector controlled by Agilent HPLC218 Chem Station Rev.A.09.03 software for data acquisition and analysis. A gradient method was used for the determination of all pro-drugs and their hydrolysis products including Apo805 on an Agilent Eclipse XDB, C18 column (part #963967-902, 150×4.6 mm, 3.5 μm) with the following chromatographic conditions:
Temperature: Ambient
Mobile phase: A=Aqueous phase: 10 mM Tris-HCl, 2 mM EDTA, pH 7.4
   B=Organic phase: Acetonitrile
   Gradient*(min-percent B): 0-5, 20-50, 25-50, 25.5-5, 30-5
Mobile phase flow rate: 1.0 mL/min
Injection volume: 50 μL
Data acquisition time: 30 min
Detection wavelength: 280 nm; 4 nm bandwidth, ref. 360 nm, 4 nm bandwidth

The chromatograms at λ=280 nm were analyzed. Peak area (mAU*s) was used for quantitation of pro-drugs, intermediates and Apo805.

Bioconversion of Apo804, and Apo838 was studied in vitro by incubation with human cryopreserved hepatocytes. HPLC analysis of the incubation mixture confirmed the formation of Apo805 from the pro-drugs (results shown in Table 2).

TABLE 2
In vitro bioconversion of H-D-Glu-(gamma-
D-Trp-OR²)-alpha-OR¹ in human hepatocytes.

| | | | Bioconversion to Apo805 |
|---|---|---|---|
| Compound ID | R¹ | R² | in human hepatocytes |
| Apo804 | PhCH₂ | Me | 31% in 4 h |
| Apo838 | Et | Et | 36% in 4 h |
| Apo840 | Me | Me | 30% in 3 h |
| Apo845 | iPr | iPr | 25% in 3 h |

### Example 26

### Caco-2 Cell Permeability Evaluation of H-D-Glu-(gamma-D-Trp-OR²)-alpha-OR¹ or H-D-Glu(D-Trp-OR²)—OR¹

Human intestinal absorption potential of H-D-Glu-(gamma-D-Trp-OR²)— alpha-OR¹ was estimated in caco-2 permeability assay.

### Cell Culture

Caco-2 cells obtained originally from ATCC were seeded onto 0.9-cm² PET filter (Becton Dickinson) at a density of 90000 cells/insert. Culture conditions were maintained for 21-28 days in 20% fetal bovine serum containing eagle's minimum essential medium enriched with non-essential amino acids. Integrity of the cell monolayers was evaluated via measurement of Lucifer Yellow paracellular apparent permeability coefficient (Papp).

### Transport Experiments

Prior to the addition of a test compound, growth medium was removed and monolayer was rinsed twice with Hank's balanced salt solution (HBSS) at 37° C. The filter inserts containing the cell monolayers were transferred to a separate 12-well) cell culture plate containing HBSS or solution of the test compound in the bottom chamber. All drug transport experiments were performed at 37° C. using 50 μM solution of the test compound in HBSS at pH 7.4. The top chamber medium volume was 1 mL and the bottom chamber medium volume was 2 mL. For every experiment, the test compound solution was added to the top (apical-to-basolateral transport, A>B) or bottom (basolateral-to-apical transport, B>A) chamber and its appearance in the opposite chamber over time was monitored. A 100 μL sample was taken from the donor chamber immediately after the addition of the compound to confirm the initial concentration of the test compound (C₀). At 30, 60, 90 and 120 min, 100 μL of supernatant sample was removed from the receiving chamber followed by the addition of 100 μL of pre-heated buffer as replenishment. At 120 min, a 100 μL supernatant sample was taken from the donor chamber to determine the concentration of compound remaining at the end of experiment. Samples were analyzed by LC-MS/MS. In case of prodrugs which undergo partial hydrolysis during the experiment, the samples were analyzed for the concentration of the prodrug and all hydrolysis products.

### Permeability Calculations

The accumulated amount of a test compound appearing in the receiving chamber over time, dQ/dt, was used to calculate the apparent permeability (Papp) using the following equation: Papp=dQ/dt×1(A×C₀), where A is the area of the filter (0.9 cm²) and C₀ is the initial concentration of the test compound in the donor chamber. For test compounds that undergo partial hydrolysis during the experiment, the total amount (in moles) of transported material was used for calculations. For each test compound, Papp values for both A>B and B>A directions were therefore calculated using the slope of the steady-state rate constant dQ/dt for the respective direction. A high absorption potential was estimated from the Papp (A>B) if the value equaled to or was higher than 1.0×10⁶ cm/s. An efflux profile was indicated if the ratio Papp (B>A)/Papp (A>B) equaled to or was higher than 2.5.

### Results

The apparent permeability was 4.28×10⁻⁶ cm/s for Apo804, 3.04×10⁻⁶ cm/s for Apo838 indicating a high permeability potential.

### Example 27

### Determination of Distribution Coefficient, D_{7.4}

MOPS buffer (50 mM, pH=7.4) and 1-octanol were used as the aqueous phase and the organic phase, respectively. The MOPS buffer and 1-octanol were mixed, and pre-saturated with each other prior to use.

In a typical experiment, an aqueous solution of Apo840 hydrochloride salt (H-D-Glu(D-Trp-O-isoamyl)-O-isoamy HCl) was prepared by weighing out 2 mg of the compound into a 5-mL volumetric flask, followed by addition of MOPS buffer (50 mM, pH=7.4) to volume. The resulting mixture was sonicated and vortexed to ensure complete dissolution. The resulting solution was analyzed by HPLC (Column: XTerra MS C₁₈, 5 μM, 4.6×250 mm; Mobile phase: A=4 mM Tris, 2 mM EDTA, pH 7.4 aqueous, B=acetonitrile; Gradient method: time in minutes—B in %: 0-5, 15-55, 25-55, 25.05-5, 30-5; Flow rate: 1 mL/min; Injection volume=2 μL; detector wavelength: 282 nm) to obtain the peak height (H_{aqu}^{I}).

One mL of this aqueous solution was pipetted out into another 10-mL test-tube and mixed with 1 mL of 1-octanol. The mixture was vortexed for 1 hour, then centrifuged at 4000 rpm for 15 minutes. The two phases were separated. Both the aqueous phase and the organic phase were analyzed by HPLC to obtain the peak heights, H_{aqu}^{F} and H_{org}^{F}. The distribution coefficient, D_{7.4}, was calculated using one or both the following equations: D_{7.4}=(H_{aqu}^{I}-H_{aqu}^{F})/H_{aqu}^{F}, or D_{7.4}=H_{org}^{F}/H_{aqu}^{F}.

The D_{7.4} of Apo840 was determined to be 3.7, and hence the logD_{7.4} was calculated to be 0.57. In a similar fashion, the logD_{7.4} of the following compounds H-D-Glu(D-Trp-OH)—O-Me (−2.22), H-D-Glu(D-Trp-O-Me)-OH (−0.89) and H-D-Glu(D-Trp-OH)—OH (−3.22) were determined.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. Furthermore, numeric ranges are provided so that the range of values is recited in addition to the individual values within the recited range being specifically recited in the absence of the range. The word “comprising” is used herein as an open-ended term, substantially equivalent to the phrase “including, but not limited to”, and the word “comprises” has a corresponding meaning. As used herein, the singular forms “a”, “an” and “the” include plural references unless the context clearly dictates otherwise. Thus, for example, reference to “a thing” includes more than one such thing. Citation of references herein is not an admission that such references are prior art to the present invention. Furthermore, material appearing in the background section of the specification is not an admission that such material is prior art to the invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

What is claimed is:

1. A compound of Formula Q1: 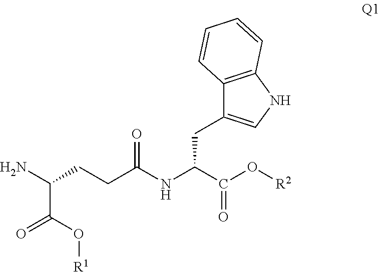
or a pharmaceutically acceptable salt thereof, wherein
R¹ and R² are independently methyl, ethyl, isopropyl, or tert-butyl; or
R¹ is benzyl and R² is methyl, ethyl, or benzyl.

2. The compound of claim 1 wherein if R¹ is benzyl or methyl, then R² is ethyl, isopropyl or tert-butyl.

3. The compound of claim 1 wherein R¹ is the same as R².

4. The compound of claim 1 wherein R¹ and R² are methyl.

5. The compound of claim 1 wherein R¹ and R² are ethyl.

6. The compound of claim 1 wherein R¹ and R² are isopropyl.

7. The compound of claim 1 wherein R¹ and R² are benzyl.

8. The compound of claim 1 wherein R¹ and R² are tert-butyl.

9. The compound of claim 1 wherein R¹ is ethyl, and R² is methyl.

10. The compound of claim 1 wherein R¹ is ethyl, and R² is isopropyl.

11. The compound of claim 1 wherein R¹ is tert-butyl, and R² is isopropyl.

12. The compound of claim 1 wherein R¹ is benzyl, and R² is ethyl.

13. A pharmaceutical formulation comprising an effective amount of a compound of Formula Q1: 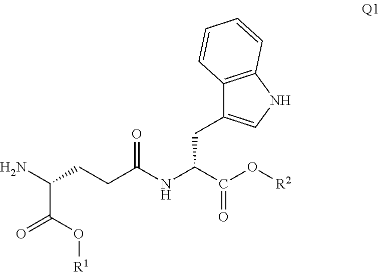
or a pharmaceutically acceptable salt thereof, wherein
R¹ and R² are independently methyl, ethyl, isopropyl, or tert-butyl; or
R¹ is benzyl and R² is methyl, ethyl, or benzyl.

14. The pharmaceutical composition of claim 13 wherein R¹ is the same as R².

15. The pharmaceutical composition of claim 13 wherein R¹ and R² are methyl.

16. The pharmaceutical composition of claim 13 wherein R¹ and R² are ethyl.

17. The pharmaceutical composition of claim 13 wherein R¹ and R² are isopropyl.

18. The pharmaceutical composition of claim 13 wherein R¹ and R² are benzyl.

19. The pharmaceutical composition of claim 13 wherein R¹ and R² are tert-butyl.

20. The pharmaceutical composition of claim 13 wherein R¹ is ethyl, and R² is methyl.

21. The pharmaceutical composition of claim 13 wherein R¹ is ethyl, and R² is isopropyl.

22. The pharmaceutical composition of claim 13 wherein R¹ is tert-butyl, and R² is isopropyl.

23. The pharmaceutical composition of claim 13 wherein R¹ is benzyl, and R² is ethyl.

24. A method of medical treatment of a subject at risk for or having psoriasis, the method comprising administering to the subject a therapeutically effective amount of a compound of Formula Q2: 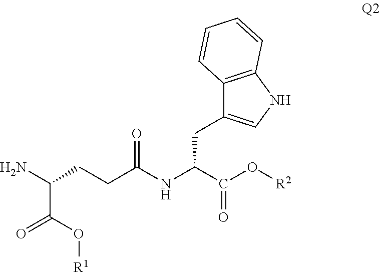
or a pharmaceutically acceptable salt thereof, wherein
R¹ is selected from the group consisting of: C₁ to C₄ alkyl and benzyl; and
R² is selected from the group consisting of: C₁ to C₄ alkyl and benzyl.

25. The method of claim 24 wherein R¹ and R² are independently methyl, ethyl, isopropyl, or benzyl.

26. The method of claim 24 wherein R¹ is the same as R².

27. The method of claim 24 wherein R¹ and R² are methyl.

28. The method of claim 24 wherein R¹ and R² are ethyl.

29. The method of claim 24 wherein R¹ and R² are isopropyl.

30. The method of claim 24 wherein R¹ and R² are benzyl.

31. The method of claim 24 wherein R¹ is ethyl, and R² is methyl.

32. The method of claim 24 wherein R¹ is ethyl, and R² is isopropyl.

33. The method of claim 24 wherein R¹ is benzyl, and R² is ethyl.
